(19) United States
(12) Patent Application Publication
(10) Pub. No.: US 2013/0267680 A1
(43) Pub. Date: Oct. 10, 2013

(54) TOTAL CHEMICAL SYNTHESIS OF UBIQUITIN, UBIQUITIN MUTANTS AND DERIVATIVES THEREOF

(71) Applicants: Huib Ovaa, Amsterdam (NL); Farid El Oualid, Amsterdam (NL); Remco Nicolaas Sebastiaan Michel Merkx, Amsterdam (NL)

(72) Inventors: Huib Ovaa, Amsterdam (NL); Farid El Oualid, Amsterdam (NL); Remco Nicolaas Sebastiaan Michel Merkx, Amsterdam (NL)

(73) Assignee: Stichting Het Nederlands kankerinstituut

(21) Appl. No.: 13/823,728

(22) PCT Filed: Sep. 15, 2011
(86) PCT No.: PCT/NL11/50623

§ 371 Date: Jun. 3, 2013

Related U.S. Application Data
(60) Provisional application No. 61/383,029, filed on Sep. 15, 2010.

(51) Int. Cl. C07K 1/04

(52) U.S. Cl. CPC C07K 1/04
USPC 530/324

(57) ABSTRACT

The present invention relates to the field of total chemical synthesis of ubiquitin and related peptides. More in particular, a method is provided of solid phase synthesis of ubiquitin, ubiquitin mutants and derivatives thereof. It was the object of the present invention to provide an approach for the total chemical synthesis of ubuiqitin, which allows for the chemical synthesis of virtually any Ub mutant and giving high overall efficiency and purity. The present inventors have surprisingly found that this object can be realized with a method relying on incorporation of special amino acid building blocks. This approach was found to allow for exceptionally high yields of up to 14% and to provide an synthetic entry into virtually any ubiquitin derivative.

### FIELD OF THE INVENTION

The present invention relates to the field of total chemical synthesis of peptides. More in particular, a method is provided of solid phase synthesis of ubiquitin, ubiquitin mutants and derivatives thereof.

### BACKGROUND OF THE INVENTION

Ubiquitin (Ub) is a highly conserved small protein that functions as a post-translational modifier, regulating a wide range of biological processes, including degradation by the proteasome, cellular localization and control of transcriptional activity and repair. It is linked to target proteins via an (iso)peptide bond between its C-terminal carboxylate and the E-amine of a lysine (Lys) residue or N-terminus of the target protein. This conjugation involves a cascade of E1, E2 and E3 enzymes, defined combinations of which trigger specific Ub modification. The E1 enzyme initiates the cascade by activating the Ub via a two-step process: formation of a Ub-adenylate, at the expense of ATP, followed by thioesterification of the adenylate with an E1 active site cysteine residue. Next, the activated Ub-thioester is transferred to an E2 conjugating enzyme by means of a trans-thioesterification with an E2 active site cysteine residue. Depending on the substrate, the Ub protein is then either transferred to a lysine residue of the target protein, directly with the help of an E3 adaptor protein, or by trans-thioesterification with an E3 active site cysteine residue.

Ubiquitin consists of 76 amino acids (8565 Da) which form a tightly-bonded and compact structure, with secondary structure elements including a mixed β-sheet (five strands and seven reverse turns), 3.5 α-helixes and a small 3₁₀-helix. Although Ub and its natural amino acid mutants can be conveniently expressed, the introduction and manipulation of (multiple) unnatural building blocks is not possible by solely biological methods. In contrast, chemical methods for the synthesis of (poly)peptides allows for virtually unlimited modifications. To date, several total chemical syntheses of Ub have been reported. These syntheses make use of solid phase peptide synthesis (SPPS) methods and can be divided into two strategies. The first is a linear SPPS approach, during which the protein is constructed in one series of peptide coupling reactions. The second approach is based on the SPPS of Ub peptide segments which are then joined together by native chemical ligation (NCL) steps. The major drawback of the lineair SPPS approach is the relatively low yield (1-4%) and large amount of impurities that make extensive purifications steps necessary and the isolation of the product a challenge. A drawback of the SPPS-NCL approach is the introduction of additional reaction and purification steps for each ligation step.

It is the object of the present invention to provide an approach for the total chemical synthesis of ubuiqitin, which allows for the chemical synthesis of virtually any Ub mutant and giving high overall efficiency and purity.

### SUMMARY OF THE INVENTION

The present inventors have surprisingly found that this object can be realized with a method relying on incorporation of special amino acid building blocks. Without wishing to be bound by any particular theory, it is assumed that these building blocks prevent aggregation and the formation of secondary structures during the elongation of the polypeptide chain while anchored to a solid phase, which is believed to constitute the main limitation in the syntheses of long and/or difficult peptides. The building blocks are used to temporarily introduce a structure breaking moiety into a polypeptide sequence. After treating the peptide with a deprotecting agent, typically TFA, during the final cleavage/deprotection step, the native dipeptide sequence is regenerated by cleavage of the amide protective group.

From the peptide sequence of ubiquitin several positions have been identified that are suitable for incorporation of the building blocks.

As will be illustrated in the examples, the present approach was found to allow for exceptionally high yields of up to 14%.

In a particularly interesting aspect of the invention the synthesis of ubiquitin mutants is provided comprising the addition of one or more ligation handles for subsequent site- and chemoselective (orthogonal) modification of the peptide. In this respect, the introduction of biophysical labels such as fluorophores or affinity labels are of special interest since they yield new ubiquitin probes with high value for research in the UPS field. In other aspects the N-terminal derivatisation of ubiquitin (mutants) is provided. Furthermore, the C-terminal derivatization of Ub is highly interesting since this can provide assay reagents for the study of, amongst others, DUBs. Moreover, Ub can be C-terminally modified in such way that it can be used in the synthesis of (non hydrolysable) poly ubiquitins for antibody generation. The present synthesis also provides the basis for the synthesis of Ubδ-thiolysine mutants that can be used for chemoselective diubiquitin synthesis. Altogether, the present methods provide an synthetic entry into virtually any ubiquitin derivative.

These and other aspects of the invention will be described in more detail and illustrated here below.

### DETAILED DESCRIPTION OF THE INVENTION

Hence, a first aspect of the present invention concerns a method of preparing ubiquitin, a ubiquitin mutant or a derivative thereof, comprising the steps of:

a) synthesizing the peptide on a solid phase by stepwise coupling of Fmoc-protected, optionally further suitably side-chain protected, amino acids, dipeptides and/or oligopeptides in a linear C-terminal to N-terminal fashion; and subsequently
b) cleaving the peptide from the solid phase and deprotecting the peptide;

wherein, in step a), at least four amino acid pairs of the ubiquitin or ubiquitin mutant sequence are added to the growing peptide chain in the form of a building block, wherein said amino acid pairs are separated from each other by at least two amino acids and are selected from the pairs at positions 6-7; 8-9; 11-12; 13-14; 21-22; 46-47; and 52-53 of the ubiquitin sequence (SEQ ID no. 1) or from the corresponding pairs of a ubiquitin mutant sequence.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

Ubiquitin (Ub) refers to the 76 amino acid, 8.5 kDa, peptide common to almost all eukaryotes, which functions to direct and control protein mechanisms, such as destruction. Ubiquitin is highly conserved among eukaryotic species: Human and yeast ubiquitin share 96% sequence identity. The sequence of human ubiquitin (SEQ ID NO. 1) is:

### MQIFVKTLTGKTITLEVEPSDTIENVKAKIQDKEGIPPDQQRLIFAGKQLEDGRTLSDY NIQKESTLHLVLRLRGG

The term ‘Ubiquitin mutant’, as used herein broadly refers to naturally occurring and non-naturally occurring polypeptides which differ from the wild-type ubiquitin sequence (SEQ ID no. 1) by minor sequence modifications, but which maintain the basic polypeptide and side chain structure of the naturally occurring form. Such sequence modifications include, but are not limited to: changes in one or a few amino acid side chains; changes in one or a few amino acids, including deletions (e.g., a truncated version of the peptide), insertions, also including the addition of N- or C-terminal amino acids, and substitutions; and changes in stereochemistry of amino acids.

A mutant herein is understood to refer to a polypeptide chain consisting of or comprising an amino acid sequence having at least 70%, preferably at least 80%, more preferably at least 90%, still more preferably at least 95%, still more preferably at least 98% and most preferably at least 98.5% amino acid sequence identity with the wild-type ubiquitin amino acid sequence (SEQ ID NO. 1), when optimally aligned, such as by the programs GAP or BESTFIT using default parameters, while preferably still displaying most or all functionality of wild-type ubiquitin. Generally, the GAP default parameters are used, with a gap creation penalty=8 and gap extension penalty=2. For proteins the default scoring matrix is Blosum62 (Henikoff & Henikoff, 1992, PNAS 89, 915-919). Sequence alignments and scores for percentage sequence identity may be determined using computer programs, such as the GCG Wisconsin Package, Version 10.3, available from Accelrys Inc., 9685 Scranton Road, San Diego, Calif. 92121-3752, USA. Alternatively percent similarity or identity may be determined by searching against databases such as FASTA, BLAST, etc.

As used herein, a mutant preferably has maintained at least some of the functionality of the naturally occurring polypeptide. Preferably functionality is either enhanced or substantially similar as the naturally occurring polypeptide. In an embodiment of the invention however mutants may be synthesized which result in impairment of one or several specific ubiquitin functionalities, e.g. proteasome binding or formation of poly Ub chains, while maintaining others. Such mutants may for example constitute valuable investigative tools.

Certain non-naturally occurring mutants are of particular interest in the context of the present invention. These include, in particular, mutants comprising insertions, additions and substitutions that can introduce or affect chemical or biological functionality, e.g. cell permeability enhancement, proteasome targeting, introduction of sites for directed chemical modifications (introduction of a so-called ‘ligation handle’), affinity tagging, etc. Preferred examples include addition of cell penetration enhancing peptide sequences such as (D-Arg)₈, Tat and penetratin; addition of affinity tag peptide sequences, such as HA and His6; addition of a proteasome targeting handle such as L4; and substitution of N- or C-terminal residues interfering with normal ubiquitin functions. Non-naturally occurring mutants of particular interest furthermore include mutants comprising certain insertions and/or substitutions that create ligation handles, especially the substitution of lysine with δ-thiolysine, δ-selenolysine, γ-thiolysine, γ-selenolysine (all as described in co-pending patent application no. PCT/NL2010/050277) or δ-azido ornithine or the substitution of leucine with photoleucine. For illustrative purposes the structural formulas of some of suitably protected δ-azido ornithine (formula (A)), γ-thiolysine (formula (B)), δ-thiolysine (formula (C)) and photoleucine (formula (D)) are shown below.

Some particularly preferred examples of non-natural ubiquitin mutants include UbG76V (SEQ ID no. 2); UbG76C (SEQ ID no. 3); UbM1C (SEQ ID no. 4); HA-Ub (SEQ ID no. 5); His6-Ub (SEQ ID no. 6); (D-Arg)8-Ub (SEQ ID no. 7); Ub-(D-Arg)8 (SEQ H) no. 8); Ub-penetratin (SEQ ID no. 9); penetratin-Ub (SEQ ID no. 10); Ub-Tat (SEQ ID no. 11); Tat-Ub (SEQ ID no. 12); Ub-L4 (SEQ ID no. 13); UbM1(OrnN₂) (SEQ ID no. 14); UbK6(OrnN₂) (SEQ ID no. 15); UbK11(OrnN₂) (SEQ ID no. 16); UbK27(OrnN₂) (SEQ ID no. 17); UbK29(OrnN₂) (SEQ ID no. 18); UbK33(OrnN₂) (SEQ ID no. 19); UbK48(OrnN₂) (SEQ ID no. 20); UbK63(OrnN₂) (SEQ ID no. 21); UbK6(6-thioK)G76V (SEQ ID no. 22); UbK11(δ-thioK) G76V (SEQ ID no. 23); UbK27(δ-thioK)G76V (SEQ ID no. 24); UbK29(6-thioK)G76V (SEQ ID no. 25); UbK33(δ-thioK) G76V (SEQ ID no. 26); UbK48(6-thioK)G76V (SEQ ID no. 27); UbK63(δ-thioK)G76V (SEQ ID no. 28), UbK6(6-thioK) (SEQ

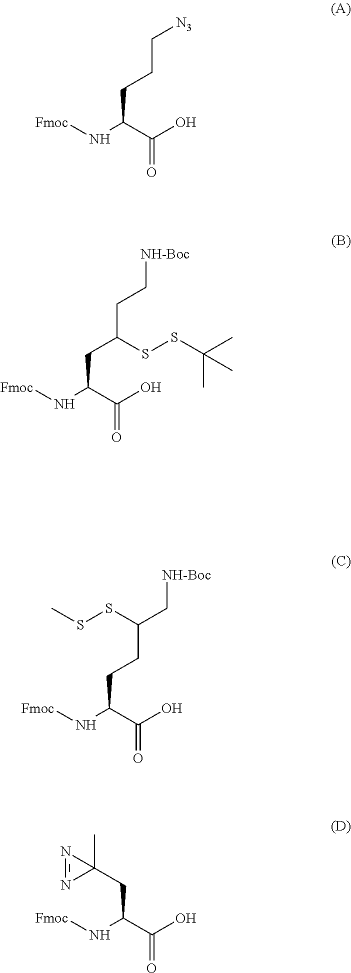

ID no. 29), UbK11(δ-thioK) (SEQ ID no. 30); UbK27(δ-thioK) (SEQ ID no. 31); UbK29(δ-thioK) (SEQ ID no. 32); UbK33(δ-thioK) (SEQ ID no. 33); UbK48(δ-thioK) (SEQ ID no. 34); and UbK63(δ-thioK) (SEQ ID no. 35), UbK48(γ-thioK) (SEQ ID no. 36), UbL43photoLeu (SEQ ID no. 37), UbL71photoLeu (SEQ ID no. 38) and UbL73photoLeu (SEQ ID no. 39), all as defined in table 1 below.

As defined above, in the present invention, four or more amino acid pairs of the amino acid sequence to be synthesized are added to the growing chain in the form of a building block. It is to be understood that, in this context, the term ‘pair’ is used herein to denote any combination of two adjacent amino acids in the peptide sequence. In this document pairs of adjacent amino acids are denoted by their position in the wild-type ubiquitin sequence of SEQ ID no. 1. The term ‘corresponding pair’, is used herein simply to identify given amino acid pairs in a ubiquitin mutant by reference to their position in the wild-type ubiquitin sequence, taking account of insertions (including addition of amino acids at the N-terminus) and deletions as compared to wild-type ubiquitin. Insertions or deletions at the N-terminal side of a given amino acid pair will increase or decrease the position number thereof, as compared to wild-type ubiquitin. As is understood by those skilled in the art the suitability of an amino acid pair for addition as a building block and the effect obtained thereby does not depend on an absolute position number but on the identity of the amino acids and their position relative to other structural elements of the ubiquitin molecule. Thus, for a given ubiquitin mutant to be synthesized in accordance with the present invention, the numerical values used to denote a given amino acid (pair) in wild-type ubiquitin, are to be increased with 1 for every insertion (including N-terminal additions) and decreased with 1 for every deletion (relative to SEQ ID no. 1), appearing at the N-terminal side of each respective pair.

In accordance with the present invention, the addition of certain pairs of amino acids as building blocks serves several functions simultaneously. First of all, these building blocks may increase salvation and minimize aggregation during peptide synthesis. Furthermore, the building blocks may serve as temporary side-chain protection for certain amino acids, especially Ser and Thr, and protect against certain side reactions, such as aspartimide formation by Asp-Gly motifs.

The present inventors have established that the separation between a building block of the invention and any Proline residue typically affects the efficiency of the synthesis. Without wishing to be bound by any particular theory, it is believed that because prolines also disrupt formation of secondary structures, it is best to avoid incorporating the special building blocks nearby a proline residue. In a preferred embodiment, a method as defined herein before is provided, wherein each amino acid pair added as an amide protected building block is separated from any proline residue by at least 4 amino acids. The present inventors furthermore have established that the separation between two building blocks of the invention also typically affects the efficiency of the synthesis. Without wishing to be bound by any particular theory, it is believed that a separation between two building blocks of 2 or more amino acids is preferably. A separation of 4 or more amino acids is particularly preferred. Furthermore, it has been established that a building block is preferably inserted before a region of hydrophobic residues. Furthermore, it was established that the yield is even further increased in case at least five amino acid pairs in the form of a building block are added. Still better results are attainable if at least six amino acid pairs are added in the form of a building block. Overall yields as high as 14% can be attained in accordance with these preferred embodiments, as will be illustrated in the examples.

Hence, in one embodiment of the invention, a method as defined herein before is provided, wherein, in step a), at least five amino acid pairs of the ubiquitin or ubiquitin mutant sequence are added to the growing peptide chain in the form of a building block, wherein said amino acid pairs are separated from each other by at least two amino acids and are selected from the pairs at positions 6-7; 8-9; 11-12; 13-14; 21-22; 46-47; 52-53; 56-57; and 65-66 of the ubiquitin sequence (SEQ ID no. 1) or from the corresponding pairs of a ubiquitin mutant sequence.

In an even more preferred embodiment, a method as defined herein before is provided, wherein, in step a), at least six amino acid pairs of the ubiquitin or ubiquitin mutant sequence are added to the growing peptide chain in the form of a building block, wherein said amino acid pairs are separated from each other by at least two amino acids and are selected from the pairs at positions 6-7; 8-9; 11-12; 13-14; 21-22; 46-47; 52-53; 56-57; and 65-66 of the ubiquitin sequence (SEQ ID no. 1) or from the corresponding pairs of a ubiquitin mutant sequence.

Most preferably, a method as defined herein before is provided, wherein, in step a), the amino acid pairs at positions 8-9; 13-14; 46-47; 52-53; 56-57; and 65-66 of the ubiquitin sequence (SEQ ID no. 1) or the corresponding pairs in a ubiquitin mutant sequence are added to the growing peptide chain in the form of a building block.

The term ‘building block’ as used herein, refers to amino-acid based peptide structure breaking derivatives that can be added to the growing peptide chain in solid phase synthesis using regular SPPS chemistry, where after the structure breaking moiety is either cleaved off or converted to yield a regular peptide structure. In accordance with the present invention the building blocks typically contain two amino acids that are joined through an alkylated amide bond or a non-amide bond. The cleaving or conversion of the structure breaking moiety can typically be performed in a single step reaction, using mild conditions and reagents such as to avoid unwanted side-reactions. In a preferred embodiment the structure breaking moiety is converted to a regular peptide bond concurrently with the deprotection of the amino acid side chains of the peptide. In a particularly preferred embodiment of the invention, a method as defined herein before is provided, wherein the building blocks are independently selected from the group of:

pseudoproline (oxazolidine) dipeptides, typically those represented by formula (I), wherein R represents an amino acid side chain, preferably the side chain of an amino acid selected from the group consisting of Ala, Asn, Asp, Gln, Glu, Gly, Ile, Leu, Lys, Phe, Ser, Trp, Tyr and Val, and R′ represents hydrogen or methyl; X represents hydrogen, branched or linear alkyl, linear or branched alkenyl or linear or branched alkynyl; preferably linear or branched C₁-C₅ alkyl, more preferably linear C₁-C₃ alkyl, most preferably methyl; and Y represents hydrogen, branched or linear alkyl, linear or branched alkenyl or linear or branched alkynyl; preferably linear or branched C₁-C₅ alkyl, more preferably linear C₁-C₃ alkyl, most preferably methyl;

dimethoxybenzyl dipeptides, typically those represented by formula (II), wherein R represents an amino acid side chain, preferably the side chain of an amino acid selected from the group consisting of Ala, Asp, Gly, Ile, Leu, and Val; and Z represents branched or linear alkyl, linear or branched alkenyl, linear or branched alkynyl, cycloalkyl, cycloalkylalkyl, cycloalkenyl, cycloalkenylalkyl, aryl, arylalkyl, heteroaryl and heteroarylalkyl, wherein each cycloalkyl-, cycloalkenyl-, aryl-, or heteroaryl-moiety may be fused to one or more additional, cycloalkyl-, cycloalkenyl-, aryl- or heteroaryl-moieties and wherein each of the aforementioned moieties may be substituted with one or more substituents selected from hydroxyl, alkoxyl, cycloalkyl, cycloalkenyl, aryl and heteroaryl, preferably from hydroxyl, methoxyl and ethoxyl; preferably Z represents methyl, ethyl, propyl, isopropyl, butyl, isobutyl, sec-butyl, tert-butyl, pentyl, isopentyl, sec-pentyl, dimethoxyethyl, diethoxyethyl, dimethoxybenzyl, dietehoxybenzyl, ethoxymethoxybenzyl, hydroxymethoxybenzyl and hydroxyethoxybenzyl; most preferably Z represents 2,4-dimethoxybenzyl or 2-hydroxy-4-methoxybenzyl; and

isoacyl dipeptides, typically those represented by formula (III), wherein R represents an amino acid side chain, preferably the side chain of an amino acid selected from the group consisting of Ala, Asn, Asp, Arg, Gln, Glu, Gly, H is, Ile, Leu, Lys, Met, Phe, Ser, Thr, Trp, Tyr and Val, and R′ represents hydrogen or methyl.

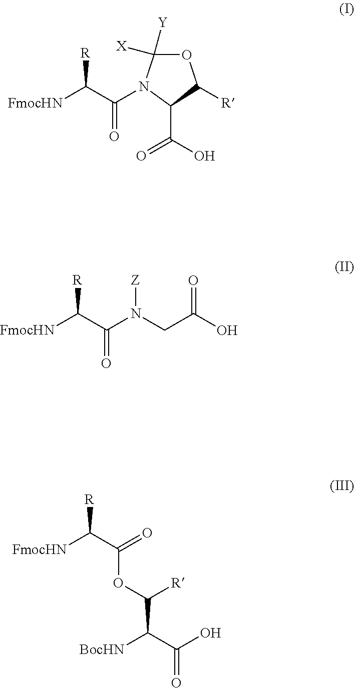

As utilized here above, the term “alkyl”, either alone or within other terms, means an alkyl moiety, preferably containing from 1 to 10, more preferably from 1 to about 8 carbon atoms and most preferably 1 to about 6 carbon atoms. The term “alkenyl” refers to an unsaturated, acyclic hydrocarbon moiety in so much as it contains at least one double bond. Such alkenyl groups typically contain from 2 to 10 carbon atoms, preferably from 2 to 8 carbon atoms and most preferably 2 to about 6 carbon atoms. The term “alkynyl” refers to an unsaturated, acyclic hydrocarbon moiety in so much as it contains one or more triple bonds, such moieties typically containing from 2 to 10 carbon atoms, preferably having from 2 to 8 carbon atoms and most preferably from 2 to 6 carbon atoms. The term “cycloalkyl” refers to carbocyclic moieties typically having 3 to 10 carbon atoms, preferably 3 to 8 carbon atoms, most preferably 5 to 8 carbon atoms. The tem “cycloalkenyl” embraces carbocyclic moieties having 3 to 10 carbon atoms and one or more carbon-carbon double bonds. Preferred cycloalkenyl moieties are “lower cycloalkenyl” radicals having 3-8 carbon atoms, more preferably 5-8. The term “aryl”, means a 5-10 membered carbocyclic aromatic ring and embraces moeities such as phenyl, naphthyl, tetrahydronaphthyl, indane and biphenyl. The term “heteroaryl” is used herein to mean a 5-10 membered carbocyclic aromatic ring containing one or more heteroatoms selected from the group consisting of N, O or S. The terms “cycloalkylalkyl”, “cycloalkenylalkyl”, “arylalkyl” and “heteroarylalkyl” embrace, respectively, the afore-defined cycloalkyl, cycloalkenyl, aryl and heteroaryl moieties attached to the amide nitrogen, i.e. of the basic moiety depicted in the formula, through an alkylene moiety, typically an alkylene moiety having 1-10, preferably 1-8, most preferably 1-6 carbon atoms, as will be understood by those skilled in the art. Ring systems containing one, two or three carbocyclic moieties which may be attached together in a pendant manner or may be fused are also embraced by the present invention. The term “fused” means that a second ring is present having two adjacent atoms in common with the first ring. The term “fused” is equivalent to the term “condensed”.

In a preferred embodiment, a method as defined herein before is provided, wherein the amide protected building blocks are independently selected from the group consisting of Fmoc-Leu-Thr(ψ^{Me,Me}pro)-OH; Fmoc-Ile-Thr(ψ^{Me,Me}pro)-OH; Fmoc-Ala-(Dmb)-Gly-OH; Fmoc-Lys(Boc)-Thr(ψ^{Me,Me}pro)-OH; Fmoc-Asp(OtBu)-Thr(ψ^{Me,Me}pro)-OH; Fmoc-Asp(OtBu)-(Dmb)-Gly-OH; Fmoc-Leu-Ser(ψ^{Me,Me}pro)-OH; Fmoc-Glu(OtBu)-Ser(ψ^{Me,Me}pro)-OH; Fmoc-Ser(tBu)-Thr(ψ^{Me,Me}pro)-OH; Boc-Thr(Fmoc-Ile)-OH; Boc-Thr[Fmoc-Lys(Boc)]-OH; Boc-Thr(Fmoc-Leu)-OH; Boc-Thr[Fmoc-Asp(OtBu)]-OH; and Boc-Thr[Fmoc-Ser(tBu)]-OH, most preferably from the group of Fmoc-Leu-Thr(ψ^{Me,Me}pro)-OH; Fmoc-Ile-Thr(ψ^{Me,Me}pro)-OH; Fmoc-Ala-(D b)-Gly-OH; Fmoc-Lys(Boc)-Thr(ψ^{Me,Me}pro)-OH; Fmoc-Asp(OtBu)-Thr(ψ^{Me,Me}pro)-OH; Fmoc-Asp(OtBu)-(Dmb)-Gly-OH; Fmoc-Leu-Ser(ψ^{Me,Me}pro)-OH; Fmoc-Glu(OtBu)-Ser(ψ^{Me,Me}pro)-OH; and Fmoc-Ser(tBu)-Thr(ψ^{Me,Me}pro)-OH.

In a preferred embodiment, a method as defined herein before is provided, wherein step a) comprises addition to the growing peptide chain of

i) Fmoc-Leu-Thr(ψ^{Me,Me}pro)-OH at amino acid positions 8-9;
ii) Fmoc-Ile-Thr(ψ^{Me,Me}pro)-OH at amino acid positions 13-14;
iii) Fmoc-Ala-(Dmb)-Gly-OH at amino acid positions 46-47;
iv) Fmoc-Asp(OtBu)-(Dmb)-Gly-OH at amino acid positions 52-53;
v) Fmoc-Leu-Ser(ψ^{Me,Me}pro)-OH at amino acid positions 56-57; and/or
vi) Fmoc-Ser(tBu)-Thr(ψ^{Me,Me}pro)-OH at amino acid positions 64-65 of the ubiquitin sequence (SEQ ID no. 1) or at the corresponding positions of a ubiquitin mutant sequence. This method may typically result in a yield as high as 14%, as will be illustrated in the examples.

Building blocks of the present invention, typically are commercially available, e.g. from Novabiochem® (part of Merck KgaA).

Solid-phase peptide synthesis or ‘SPPS’ refers to the direct chemical synthesis of peptides, wherein an insoluble polymeric support is used as an anchor for the growing peptide chain, which is typically built up one amino acid at a time. The free N-terminal amine of a solid-phase attached peptide is coupled to an N-protected amino acid unit. This unit is then deprotected, revealing a new N-terminal amine to which a further amino acid unit may be attached. The general principle of SPPS is one of repeated cycles of such coupling-wash-deprotection-wash steps, adding, typically one amino acid at a time, until the peptide of the desired sequence and length has been synthesized. As will be understood by those skilled in the art it is possible, in principle, to couple N-protected peptides instead of single amino acids to the growing chain in one or more elongation cycles. The present invention also encompasses methods wherein one or more larger N-protected peptides, or oligopeptides, typically having a length of up to 20 amino, preferably up to 10 amino acids, more preferably up to 5 amino acids, still more preferably up to 4 amino acids are added to the growing chain. In a particularly preferred embodiment, a method as defined herein before is provided, wherein step a) comprises stepwise coupling amino acids, dipeptides and/or tripeptides, preferably amino acids and/or dipeptides to the growing peptide chain. In a most preferred embodiment of the invention, step a) comprises stepwise coupling of single amino acids or building blocks to the growing peptide chain.

Preferably, in accordance with the present invention, the growing peptide is anchored to the resin or resin handle through the terminal carboxyl group. Nevertheless the use of certain linkers allowing for anchoring of the growing peptide-chain via a side-chain residue, is also envisaged and may even be preferred, especially in case the peptide is to be C-terminally modified after synthesis, as will be described herein below.

The solid phase for SPPS typically is a solid, non-soluble support material. For the purposes of the present invention, such a solid phase material comprises sites for anchoring of a first amino acid (or peptide). Such functional sites for anchoring of the peptide are termed linkers. If need be, other linker moieties such as e.g. more specialized, for instance more acid-labile, linkers may be grafted to the first, integral linkers on the premade solid phase, which is often then referred to as a ‘handle’. Polymeric organic resin supports are the most common type of solid phase material, typically comprising highly solvated polymers with an equal distribution of functional groups. Examples include Polystyrene (PS); Polyacrylamide (PA); polyethylene glycol (PEG); PEG-Polystyrene (PEG-PS) or PEG-Polyacrylamide (PEG-PA); and other PEG-based supports. The invention is not particularly limited with respect to the solid phase material. The so-called Wang resin (4-Benzyloxybenzyl Alcohol resin) and PAM resin (4-hydroxymethyl-phenylacetamidomethyl), are particularly suitable solid phase materials for methods of the present invention. Other suitable examples include, but are not limited to: PEG-HMPB (cross-linked PEG functionalized with 4-(4-Hydroxymethyl-3-methoxyphenoxy)butyric acid); Rink amide resin (4-(2′,4′-Dimethoxyphenyl-Fmoc-aminomethyl-phenoxy-resin); and Merrifield resin (copolymer of styrene and chloromethylstyrene cross-linked with divinylbenzene). Solid support materials should meet several requirements, besides being chemically inert and able to withstand the conditions of synthesis: solid phase particles are preferably of conventional and uniform size, mechanically robust, easily filterable and highly accessible to the solvents allowing the penetration of the reagents and the enlargement of the peptide chain within its microstructure. Resins as used in the present invention are typically of standard mesh size, which is about 50-500 mesh, more preferably 100 to 400 mesh.

As stated above, the present method concerns so-called ‘Fmoc SPPS’ methods, wherein Fmoc (Fluorenylmethyloxycarbonyl) N-protected amino acids and peptides are added to the growing chain. Fmoc protection in solid phase peptide synthesis has significant advantages because its removal involves very mild basic conditions (e.g. piperidine solution), such that it does not disturb the acid labile linker between the peptide and the resin. Fmoc N-protected amino acids are commercially available. Furthermore, reactions to produce Fmoc N-protected amino acids or peptides are common general knowledge for those skilled in the art.

Each incoming amino acid that is added to the growing peptide chain is preferably also protected, where suitable, with a side-chain protecting group, which is typically acid-labile. Protection groups suitable for this purpose are well known in the art. Commonly employed carboxy-protection groups for Glutamine and Aspartic acid are e.g. Mpe, O-1-Adamantyl, O-benzyl and even simply alkyl esters may be used, though less common. For sake of ease, typically and preferably tert-butyl groups are used. Tyrosine may typically be protected by protection groups such as tert-butyl ether or Z- or more preferably 2-Bromo-Z-esters. It is equally possible to use tritylalkohol protection groups such as 2-chloro-trityl or 4-methoxy or 4,4′ methoxy-trityl groups. Preferably, a trityl or a tert-butyl (tBu) protection group is used, most preferably a tBu protection group, meaning the tyrosyl side chain is modified to a tertiary-butyl ether. The tBu group is only efficiently removed under strongly acidic condition. Suitable Arginine protective groups include 2,2,4,6,7-pentamethyldihydrobenzofuranyl-5-sulfonyl (Pbf), adamantyloxy-carbonyl and isobornyl-oxy-carbonyl, 2,2,5,7,8-pentamethylenchromanesulfonyl-6-sulfonyl (Pmc), 4-methoxy-2,3,6-trimethylbenzenesulfonyl (Mtr) and its 4-tert.butyl-2,3,5,6-tetramethyl homologue (Tart) or Boc, which are only cleaved under strongly acidic conditions. Preferably, Pbf, Pmc, Mtr, most preferably Pbf is used. Upon global deprotection of side chains under strongly acidic conditions, in usually aequeous medium, bystander-alkylation of deprotected tyrosine is not observed with Pmc, Mtr and Pbf. Serine and, Threonine typically may be protected by e.g. tert-butyl or trityl, most preferably tert-butyl. Other modes of protection are equally feasible, e.g. with benzyl, though less preferred since eventually requiring removal under less desirable condition. Similar considerations apply to protection of Lysine; typically and preferably, Lys is protected with Boc. Tryptophan must not necessarily be protected during solid-phase synthesis, though protection with typically Boc is evisaged. As regards side chain protection groups, the afore said is valid both for the natural L-amino acids as well as for their D-homologues.

Coupling reagents for Fmoc peptide synthesis are well-known in the art. Coupling reagents may be mixed anhydrides, (e.g. propane phosphonic acid anhydride or *T3P′) or other acylating agents such as activated esters or acid halogenides (e.g. isobutyl-chloroformiate or ‘ICBF’), or they may be carbodiimides (e.g. 1-ethyl-3-(3-dimethylaminopropyl)-carbodiimide, diisopropyl-carbodiimide, dicylcohexyl-carbodiimide), activated benzotriazine-derivatives (e.g. 3-(diethoxyphosphoryloxy)-1,2,3-benzotriazine-4(3H)-one or ‘DEPBT’) or uronium or phosphonium salt derivatives of benzotriazol. In view of best yield, short reaction time and protection against racemization during chain elongation, it is preferred that the coupling reagent is selected from the group consisting of uronium salts and phosphonium salts of benzotriazol capable of activating a free carboxylic acid function along with that the reaction is carried out in the presence of a base. Suitable and likewise preferred examples of such uronium or phosphonium coupling salts are e.g. HBTU (O-1H-benzotriazole-1-yl)-N,N,N′,N′-tetramethyl uronium hexafluorophosphate). BOP (benzotriazole-1-yl-oxy-tris-(dimethylamino)-phosphonium hexafluorophosphate), PyBOP (Benzotriazole-1-yl-oxy-tripyrrolidinophosphonium hexafluorophosphate), PyAOP, HCTU (O-(1H-6-chloro-benzotriazole-1-yl)-1,1,3,3-tetramethyluronium hexafluorophosphate), TCTU (O-1H-6-chloro benzotriazole-1-yl)-1,1,3,3-tetramethyluronium tetrafluoroborate), HATU (O-(7-azabenzotriazol-1-yl)-1,1,3,3-tetramethyluronium hexafluorophosphate), TATU (O-(7-azabenzotriazol-1-yl)-1,1,3,3-tetramethyluronium tetrafluoroborate), TOTU (O-[cyano(ethoxycarbonyl)methyleneamino]-N,N,N′,N′-tetramethyluronium tetrafluoroborate), HAPyU (O-(benzotriazol-1-yl)oxybis-(pyrrolidino)-uronium hexafluorophosphate.

For coupling of the Fmoc amino acids to the peptide, the carboxyl group is usually activated. This is important for speeding up the reaction. There are two main types of activating groups: carbodiimides and triazolols. The use of these activating coupling additives is particularly preferred when using the highly activating uronium or phosphonium salt coupling reagents. Most preferably the coupling additive is a N-hydroxy-benzotriazol derivative (or 1-hydroxy-benzotriazol derivative) or is an N-hydroxy-benzotriazine derivative. Suitable examples include. N-hydroxy-succinimide, N-hydroxy-3,4-dihydro-4-oxo-1,2,3-benzotriazine (HOOBt), 1-hydroxy-7-azabenzotriazole (HOAt) and N-hydroxy-benzotriazole (HOBt). N-hydroxy-benzotriazine derivatives are particularly preferred, in a most preferred embodiment, the coupling reagent additive is hydroxy-3,4-dihydro-4-oxo-1,2,3-benzotriazine. Most common carbodiimides are dicyclohexylcarbodiimide (DCC) and diisopropylcarbodiimide (DIC).

Activation of the Fmoc amino acid is typically done in the presence of a base reagent. Preferably, the base reagent is a weak base whose conjugated acid has a pKa value of from pKa 7.5 to 15, more preferably of from pKa 7.5 to 10, and which base preferably is a tertiary, sterically hindered amine. Examples of such and further preferred are Hunig-base (N,N-diisopropylethylamine; DIPEA), N,N′-dialkylaniline, 2,4,6-trialkylpyridine, 2,6-trialkylpyridine or N-alkyl-morpholine with the alkyl being straight or branched C1-C4 alkyl, more preferably it is N-methylmorpholine (NMM) or collidine (2,4,6-trimethylpyridine), most preferably it is collidine.

In an embodiment of the invention, chaotropic salts (CuLi, NaClO₄, KSCN) and mixtures of solvents suchs as N,N-dimethylformamide, trifluoroethanol, dimethylacetamide and N-methylpirrolidone may typically be used to improve the efficiency of coupling

The amount of the various reactants in the coupling reaction can and will vary greatly. Reagents are typically used in large excess to speed-up the reaction and drive it to completion. Typically the amount of solid support to the amount of Fmoc-amino acid will be a molar ratio ranging from about 1:1 to 1:5. In one embodiment, the amount of solid support to the amount of Fmoc-amino acid to the amount of activating compound is a molar ratio of about 1:4. The reaction conditions for the coupling steps, such as reaction time, temperature, and pH may vary without departing from the scope of the invention. The coupling temperature is usually in the range of from 15 to 30° C., especially where using phosphonium or uronium type coupling reagents. Typically, a temperature of about 20 to 25° C. is applied for coupling.

A range of color tests for the qualitative monitoring of the coupling reaction has been developed. Progress of amino acid couplings can be followed using ninhydrin, or p-chloranil test. The ninhydrin solution turns dark blue (positive result) in the presence of a free primary amine but is otherwise colorless (negative result). The p-chloranil solution will turn the solution dark black or blue in the presence of a primary amine if acetaldehyde is used as the solvent or in the presence of a secondary amine, if acetone is used instead; the solution remain colorless or pale yellow otherwise.

The N^{α} Fmoc is typically cleaved under very mild basic conditions. The standard reagent for Fmoc-deprotection in solid phase peptide synthesis is piperidine, typically 20%, in DMF or NMP. Further examples of suitable bases, include DBU, DBN and morpholine. Although the invention is not particularly limited in this respect, it is preferred to employ a mixture comprising piperidien and DMF or NMP. Deprotection can be monitored by UV absorbance of the runoff, a strategy which is also employed in automated synthesizers.

Once the final amino acid has been added, the polypeptide may be cleaved from the solid support with a mild acid in the presence of appropriate scavengers to yield a peptide-alkylamide. In general, the solid support will be treated with trifluoroacetic acid (TFA) in the presence of appropriate scavengers. The choice of scavengers is dependent on the amino acid sequence of the peptide. These scavengers include phenol, water, 1,2-ethanedithiol, and triisopropylsilane. In certain embodiments it may be desirable to deprotect all of the amino acids, or selectively deprotect certain amino acids, or to deprotect the amino acids while leaving the peptide covalently conjugated to the solid support. By varying the concentration of the mild acid, either a fully or partially protected peptide secondary amide may be released from the solid support. The amount of TFA typically used for cleavage of the protected peptide from the solid support may range from about 1% to about 10% (v/v). More typically the amount of TFA used for cleavage of the protected peptide from the solid support may range from about 3% to about 5% (v/v). It is possible to use photocleavable linkers such as for instance a carboxamide generating, photocleavable linker.

In another preferred embodiment, the solid phases of the present invention allows of cleavage of peptide from a solid phase under strongly acidic conditions. By definition, according to the present invention, a strongly acidic condition as being opposed to a weakly acidic condition typically means applying at least 50% (v/v) TFA in the solvent. Further, conversely, a protection group requiring strongly acidic condition for removal is a protection group that is removed, typically, using 80% TFA or more. Preferably, the use of protection groups that require stronger acids, such as HF, is avoided. A weakly acidic condition is defined by having 0.01% (v/v) to <50% TFA, preferably having 0.1% to 30% TFA. The term ‘acid-labile’ typically refers to essentially quantitative cleavage in 2-10% TFA at ambient temperature for at least an hour.

In a preferred embodiment, a method as defined herein before is provided, wherein step b) comprises cleaving the peptide from the solid phase material using weakly acidic conditions, and wherein the method comprises a separate step of removing the protective groups from the ubiquitin, ubiquitin mutant or derivative thereof using strongly acidic conditions. This embodiment allows for further selective modification of the peptide in solution, as will be explained in more detail hereafter.

In another preferred embodiment, a method as defined herein before is provided, wherein step b) comprises deprotecting the peptide concurrently with cleaving of the peptide from the solid phase support, using strongly acidic conditions.

In accordance with the present invention, SPPS can performed in different ways. There are manual and automated systems available for small and large scale synthesis. Typically, all operations described, namely coupling, deprotecting and final removal are conducted in the same recipient, requiring several washing steps.

In addition to variations in the amino acid sequence of ubiquitin and the introduction of unnatural amino acid building blocks, the here described methodology also allows for synthesis of derivatives of the ubiquitin or ubiquitin mutants, typically comprising coupling of a ligand to an amino acid side chain, the N-terminus and/or the C-terminus.

As used herein the terms ‘ubiquitin derivative’ and ‘ubiquitin mutant derivative’ refer to products comprising a ubiquitin or ubiquitin mutant peptide chain as defined above, further comprising one or more C-terminal, N-terminal and/or orthogonal ligands. Such ligands may, in principle, be of any nature, including peptides or proteins, lipis, carbohydrates, polymers and organic or inorganic agents. The introduction of the ligand typically introduces or affects a particular biological or chemical function. Particularly interesting examples include the introduction of detectable labels and tags, introduction of electrophilic traps, introduction of chemical ligation moieties, etc. Hence, in a preferred embodiment, a method as defined herein before is provided, wherein said one or more ligands are selected from the group of fluorophores, affinity labels, biophysical labels, chelating agents, complexing agents and epitope tags, preferably from the group of fluorescein (formula (E)), TAMRA (formula (F)), DOTA (formula (G)), AMC (formula H)), propargylamine (formula (I)), VME (formula (J)) and SEt (formula (K)). Such ligands are typically known to those skilled in the art and their introduction at a desired site can be accomplished using reagents and conditions that are generally known. Examples of particularly preferred derivatives include CF-Ub, TAMRA-Ub, DOTA-Ub, Ub-PA, Ub-VME, Ub-AMC, Ub-SEt, Ub-Rh110Gly, as defined in table 2 below.

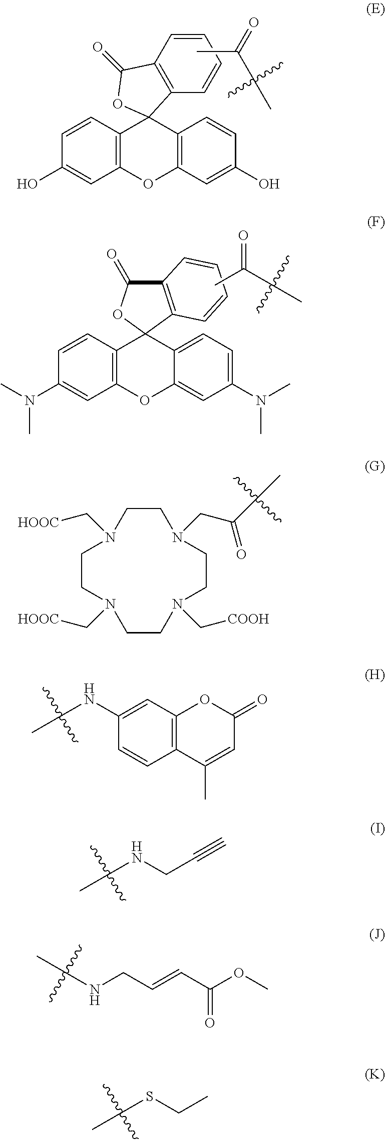

In a preferred embodiment, a method as defined herein before is provided, comprising ligation of a ligand to a reactive amino acid side chain and/or the N-terminal amine moiety of the ubiquitin or ubiquitin mutant before step b), i.e. while the peptide is still anchored to the resin. Preferably such modifications are performed while the synthesized peptide is still side chain protected. As will be understood by those skilled in the art highly selective ligation processes are conceivable by appropriate selection of side chain protecting groups. Nevertheless, as will be understood by those skilled in the art, methods wherein N-terminal and/or orthogonal derivatisation is performed after step b) are also within the general scope of the invention.

For illustrative purposes the synthesis of a series of ubiquitin derivatives that are N-terminally labeled with fluoresceine, TAMRA (fluorophores), biotin (affinity label) and DOTA (chelating agent that is able to form stable complexes with metals such as radionuclides for imaging and therapy) is described in the examples below.

The present method is particularly suitable for orthogonal derivatisation by inclusion of so-called orthogonal ligation handles, and subsequent coupling of the desired ligand(s). Such a process is usually referred to as ‘(covalent) site-specific modification’. Hence, in one particularly preferred embodiment of the invention, the method involves the step of producing a ubiquitin mutant comprising addition to the growing peptide chain, either by insertion or substitution, one or more orthogonal ligation handles, preferably an unnatural amino acid, more preferably an unnatural amino acid selected from the group of δ-thiolysine, δ-selenolysine, γ-thiolysine, γ-selenolysine (all as described in co-pending patent application no. PCT/NL2010/050277) and 6-azido ornithine, said method further comprising the step of covalently attaching one or more ligands via said ligation handle, following step a) or step b). In another embodiment however said orthogonal ligation handle to be added by insertion or substitution may be a natural amino acid, preferably cysteine. Cysteine derivatization is typically sufficiently specific and a single cysteine residue can usually be introduced without affecting the function of the protein. Methods of site-specific modification of cystein are known to those skilled in the art.

Since in conventional SPPS protocols the growing peptide chain is attached to the solid support via its C-terminus, C-terminal modifications of synthetic peptides are usually more complex than N-terminal or orthogonal modifications. Nonetheless, in the literature several methods for the C-terminal modification of synthetic peptides are described. These methods mostly rely on the use of safety-catch linkers such as the sulfonamide linker and the aryl-hydrazine linker, anchoring of the growing peptide-chain via a side-chain residue, use of the backbone amide linker (BAL), or modification of the protected peptide precursor in solution. The latter method would be the most straightforward. In accordance with this embodiment a method is provided as defined herein before, wherein step h) comprises cleavage of the polypeptide from the solid phase resin under mild conditions, thereby leaving the N-terminal and side chain protecting groups intact, followed by C-terminal derivatization of the protected peptide in solution. The free C-terminus can be modified, e.g. with suitable amine, hydroxyl, or thiol nucleophiles using standard coupling reagents in solution. Subsequently the modified peptides can be deprotected, typically under stongly acidic conditions and be worked up as described here after. For illustrative purposes the synthesis of ubiquitins that are C-terminally modified with propargylamine (handle for CuAAC), VME (electrophilic trap, AMC (fluorophore) and mercaptoethane (to obtain a thioester for NCL) is described below.

The inventors found that the orthogonal derivatisation described before is particularly suitable for the preparation of diubiquitin derivatives. In a preferred embodiment of the invention the preparation of diubiquitin conjugates is provided using on resin conjugation of two ubiquitin polypeptides, comprising the steps of

preparing fully side-chain protected ubiquitin with a free C-terminal carboxylic acid on a resin that allows the synthesis of a partially protected peptide with a free C-terminal carboxylic acid, such as trityl type resin, photocleavable resin, hydrazine type resin, safety catch resin, etc.;

preparing, separately, resin bound fully protected ubiquitin which contains a lysine residue that is orthogonally protected on the ε-amine, e.g. using orthogonal protective groups such as monomethoxytrityl (Mmt), trityl (Tr), 4-methyltrityl (Mtt), Alloc, Dde, ivDde, Z, Adpoc or photocleavable groups;

selective removal of said lysine side-chain protecting group;

coupling the partially protected ubiquitin with a free C-terminal carboxylic acid and the partially protected ubiquitin with a free lysine side-chain, using standard peptide coupling methods, yielding fully protected resin bound diubiquitin conjugate, which can be simultaneously deprotected and released from the resin affording the desired isopeptide linked diubiquitin conjugate.

A schematic representation of the process of this particular embodiment can be found in FIG. 1.

In a preferred embodiment, a method as defined herein before is provided, comprising a step c) following step b), said step c) comprising folding of the crude protein or derivative, e.g. by dialysis or dilution of a highly concentrated DMSO stock into water or buffer, and/or purifying the crude or folded protein, typically using standard methods such as reversed phase HPLC, size exclusion chromatography or cation exchange chromatography.

As will be understood by those skilled in the art, intermediate products are obtained during the various steps of the method described herein before are also encompassed by the scope of the present invention. Hence, another aspect of the concerns a substance selected from ubiquitin, ubiquitin mutants and derivatives thereof, wherein at least four amino acid pairs of the chain have been replaced with a corresponding building block, wherein said amino acid pairs are separated from each other by at least two amino acids and are selected from the pairs at positions 6-7; 8-9; 11-12; 13-14; 21-22; 46-47; and 52-53 of the ubiquitin sequence (SEQ ID no. 1) or from the corresponding pairs of a ubiquitin mutant sequence. A preferred embodiment provides such a substance selected from ubiquitin, ubiquitin mutants and derivatives thereof, wherein at least five amino acid pairs of the ubiquitin or ubiquitin mutant sequence are replaced with a building block, wherein said amino acid pairs are separated from each other by at least two amino acids and are selected from the pairs at positions 6-7; 8-9; 11-12; 13-14; 21-22; 46-47; 52-53; 56-57; and 65-66 of the ubiquitin sequence (SEQ ID no. 1) or from the corresponding pairs of a ubiquitin mutant sequence. A more preferred embodiment provides such a substance selected from ubiquitin, ubiquitin mutants and derivatives thereof, wherein at least six amino acid pairs of the ubiquitin or ubiquitin mutant sequence are replaced with a building block, wherein said amino acid pairs are separated from each other by at least two amino acids and are selected from the pairs at positions 6-7; 8-9; 11-12; 13-14; 21-22; 46-47; 52-53; 56-57; and 65-66 of the ubiquitin sequence (SEQ ID no. 1) or from the corresponding pairs of a ubiquitin mutant sequence. Still more preferably, a substance selected from ubiquitin, ubiquitin mutants and derivatives thereof is provided, wherein the amino acid pairs at positions 8-9; 13-14; 46-47; 52-53; 56-57; and 65-66 of the ubiquitin sequence (SEQ ID no. 1) or the corresponding pairs in a ubiquitin mutant sequence are replaced with a building block. In a most preferred embodiment, a substance selected from ubiquitin, ubiquitin mutants and derivatives thereof as defined herein before is provided, comprising a Leu-Thr(ψ^{Me,Me}pro) building block at amino acid positions 8-9; an Ile-Thr(ψ^{Me,Me}pro) building block acid positions 13-14; an Ala-(Dmb)-Gly building block at amino acid positions 46-47; an Asp(OtBu)-(Dmb)-Gly building block at amino acid positions 52-53; a Leu-Ser(ψ^{Me,Me}pro) building block at amino acid positions 56-57; and a Ser(tBu)-Thr(ψ^{Me,Me}pro) building block at amino acid positions 64-65 of the ubiquitin sequence (SEQ ID no. 1) or at the corresponding positions of the ubiquitin mutant sequence.

### DESCRIPTION OF THE FIGURES

FIG. 1: Schematic representation of the synthesis of diubiquitin conjugates on solid phase. PG=protecting group; X=orthogonal protecting group. i) selective cleavage of partially protected ubiquitin from resin; ii) selective removal of orthogonal protecting group; iii) amide formation; iv) deprotection and cleavage from resin.

FIG. 2: A) A & C) LC and MS profile of commercial Ub. B and D) LC and MS profile of crude synthetic Ub.

FIG. 3: Anti-Ub western blot of ubiquitin ligase assay with synthetic Ub (left) and expressed Ub (right). E1=Uba1 (500 nM), various E2s (2 μM), E3=Triad1 (1 μM), Ub (15 μM), ATP (3 mM), 30° C., 2½ h. UbCH5c forms mixed chains, E2S forms K11 linked chains, E2-25K forms K48 linked chains and Ubc13-mms2 forms K63 linked chains. The negative controls are the reactions without E1 and without E2.

FIG. 4: Circular dichroism measurement of native ubiquitin (black) versus synthetic DMSO-folded ubiquitin (grey).

FIG. 5: Hydrolysis of fluorogenic Ub derivatives by the deubiquitinating enzymes HAUSP/USP7 and UCH-L3. All assays contained 1 nM of enzyme, substrate concentration was varied. A Commercial UbAMC+USP7/HAUSP; B Synthetic UbAMC+USP7/HAUSP; C Michaelis-Menten kinetics comparison of commercial and synthetic UbAMC with USP7 shows identical kinetics; D Synthetic UbAMC+UCH-L3; E Synthetic UbRh110Gly+USP7; F Synthetic UbRh110Gly+UCH-L3;

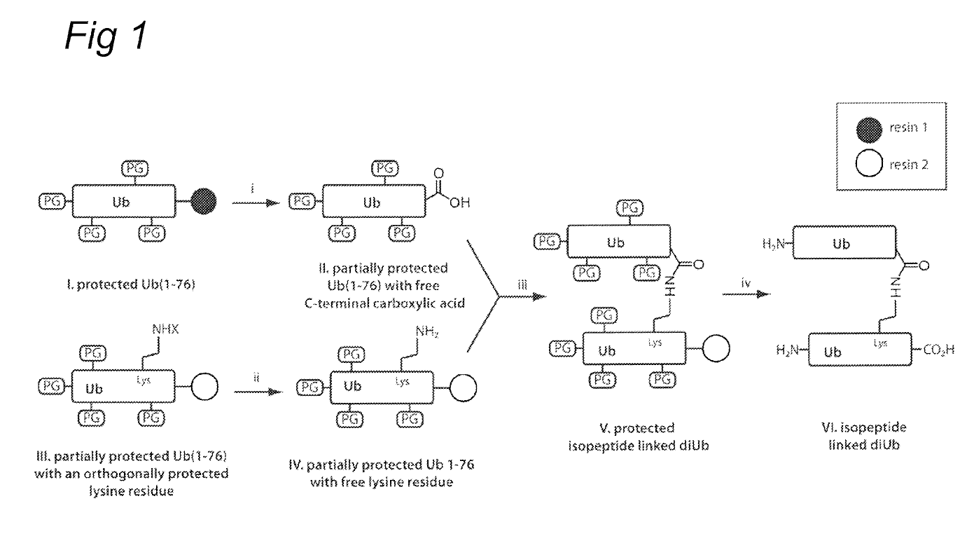

### EXAMPLE 1

### Chemical Synthesis of Ubiquitin and Ub-Derivatives Materials & Methods

### Reagents

General reagents were obtained from Sigma Aldrich, Fluka and Acros and used as received. (5R)-5-hydroxy-L-lysine dihydrochloride monohydrate was purchased from Sigma Aldrich. Solvents were purchased from BIOSOLVE or Aldrich and, where necessary, dried over molecular sieves (4 Å for DCM, DMF and 3 Å for MeOH). Peptide synthesis reagents were purchased from Novabiochem. Analytical thin layer chromatography was performed on aluminium sheets precoated with silica gel 60 F²⁵⁴ using 20% ninhydrin in ethanol and heating by a heatgun. Column chromatography was carried out on silica gel (0.035-0.070 mm, 90 Å, Acros). Nuclear magnetic resonance spectra (¹H-NMR, ¹³C-NMR and COSY) were determined in MeOD-d₄ (¹H δ 4.87 ppm; ¹³C δ 49.15 ppm) using a Bruker ARX 400 Spectrometer (¹H: 400 MHz, ¹³C: 100 MHz) at 298 K, unless indicated otherwise. Peak shapes in NMR spectra are indicated with the symbols ‘d’ (doublet), ‘dd’ (double doublet), ‘s’ (singlet) triplet and ‘m’ (multiplet). Chemical shifts (δ) are given in ppm and coupling constants J in Hz. LC-MS measurements were performed on a system equipped with a Waters 2795 Seperation Module (Alliance HT), Waters 2996 Photodiode Array Detector (190-750 nm), Waters Alltima C18 (2.1×100 mm, 3 μm), Waters Symmetry300™ C4 (2.1×100 mm, 3.5 μm) or Phenomenex Kinetex C18 (2.1×50, 2.6 μm) and LCT™ Orthogonal Acceleration Time of Flight Mass Spectrometer. Samples were run using 2 mobile phases: A=1% CH₃CN, 0.1% formic acid in water and B=1% water and 0.1% formic acid in CH₃CN. Data processing was performed using Waters MassLynx Mass Spectrometry Software 4.1 (deconvulation with Maxent1 function).

### LC-MS Programs

Program 1:

Waters AtlantisT3™ C18, 2.1×100 mm, 3 μM); flow rate=0.4 mL/min, runtime=10 min, column T=40° C. Gradient: 0-2 min: 5% B; 2-5 min: [symbol] 95% B; 5-7 min: 95% B.

Program 2:

Waters Symmetry300™ C4, 2.1×100 mm, 3.5 μM; flow rate=0.2 mL/min, runtime=30 min, column T=40° C. Gradient: 0-2 min: [symbol] 5% B; 2-3 min: 10% B; 3-17 min: [symbol] 90% B; 17-30 min: [symbol] 95% B.

Program 3:

Phenomenex Kinetex C18, (2.1×50 mm), 2.6 μM); flow rate=0.8 mL/min, runtime=6 min, column T=40° C. Gradient: 0-0.5 min: 5% B; 0.5-4 min: [symbol] 95% B; 4-5.5 min: 95% B.

### Fmoc SPPS Strategy

SPPS was performed on a Syro II MultiSyntech Automated Peptide synthesizer using standard 9-fluorenylmethoxycarbonyl (Fmoc) based solid phase peptide chemistry at 25 μmol scale using fourfold excess of amino acids relative to pre-loaded Fmoc amino acid Wang type resin (0.2 mmol/g, Applied Biosystems®) or pre-loaded Fmoc amino acid trityl resin (0.2 mmol/g, Rapp Polymere GmbH). Single couplings were performed in NMP for 40 min using PyBOP (4 equiv) and DiPEA (8 equiv) as coupling regents. The following protected amino acid, pseudoproline and DMB building blocks were used during ubiquitin synthesis: Fmoc-L-Ala-OH, Fmoc-L-Arg-(Pbf)-OH, Fmoc-L-Asn(Trt)-OH, Fmoc-L-Asp(OtBu)-OH, Fmoc-L-Gln(Trt)-OH, Fmoc-L-Glu(OtBu)-OH, Fmoc-L-Gly-OH, Fmoc-L-His(Trt)-OH, Fmoc-L-Ile-OH, Fmoc-L-Leu-OH, Fmoc-L-Lys(Boc)-OH, Fmoc-L-Met-OH; Fmoc-L-Phe-OH; Fmoc-L-Pro-OH; Fmoc-L-Ser(tBu)-OH; Fmoc-L-Thr(tBu)-OH, Fmoc-L-Tyr(tBu)-OH, Fmoc-L-Val-OH, Fmoc-L-Ser(tBu)-L-Thr(Ψ^{Me,Me}pro)-OH, Fmoc-L-Leu-L-Ser(Ψ^{Me,Me}pro) OH, Fmoc-L-Ile-L-Thr(Ψ^{Me,Me}pro)-OH: Fmoc-L-Leu-L-Thr(Ψ^{Me,Me}pro)-OH, Fmoc-L-Asp(OtBu)-(Dmb)Gly-OH and Fmoc-L-Ala-(Dmb)Gly-OH. All amino acid and building blocks were dried overnight under high vacuum prior to use. Fmoc removal was achieved with 20% piperidine in NMP (3×1.0 mL, 2×2 and 1×5 min). Capping was performed with a mixture of Ac₂O/DiPEA/HOBt in NMP at 0.5M, 0.125M and 0.015M respectively (3×1.2 mL, 2×2 and 1×5 min). After the first 30 cycles the coupling time was extended to 60 minutes and Fmoc deprotection was extended to 4×3 minutes. The polypeptide sequence was detached from the resin and deprotected by treatment with TFA/H₂O/Phenol/TIS 90.5/5/2.5/2 v/v/v/v for 3 h followed by precipitation with cold Et₂O/n-pentane 3:1 v/v and finally lyophilized from H₂O/MeCN/AcOH 65:25:10 v/v/v.

### Folding and Purification of Synthetic Ubiquitin (Mutants)

The crude Ub (mutant) is folded by taking it first up in a minimal amount of warm DMSO and then diluting the DMSO solution with 50 mM NaOAc pH 4.5 the final DMSO concentration is kept as low as possible (2-10%). Next, the folded peptide is purified by cation chromatography using a MonoS column and a 0 [symbol] 1 M NaCl gradient in 50 mM NaOAc pH 4.5. Pure fractions are analysed by LC-MS and the 50 mM NaOAc pH 4.5 buffer containing ±0.18 M NaCl is exchanged for milliQ over a 3 kDa cutoff spin-column. The Ub (mutant) in milliQ is then lyophilized.

### General Method for the N-Terminal Modification of Ub

The Ub(1-76) peptide sequence with a free N-terminus was synthesized on a Wang resin following the general procedure. For the modification reaction, a solution of the label (10 equiv), DIC (10 equiv) and HOBt (10 equiv) in NMP (800 μL) was incubated for 5 min and added to the resin-bound peptide (1 equiv). The mixture was gently shaken for 3 h at room temperature before the resin was filtered and washed with NMP, DCM and Et₂O. Post-modification work-up including cleavage/deprotection, lyophilization and purification by cation chromatography were performed according to the general procedure.

### General Method fbr the C-Terminal Modification of Ub

The Ub(1-75) peptide sequence was synthesized on a trityl resin following the general procedure except for the final methionine residue 0 which was introduced as the corresponding Boc derivative. The resin bound polypeptide was treated with 5 mL of DCM/HFIP (4:1 v/v) for 30 min and filtered. The resin was rinsed with DCM (3×5 mL) and the combined filtrates were concentrated in vacuo. The partially protected peptide residue (1 equiv) was redissolved in DCM and reacted with PyBOP (5 equiv) and an excess of the nucleophile and TEA. The reaction mixture was stirred over night at room temperature. The solvent was removed in vacuo and the residue was treated with TFA/H₂O/TiS (95:2.5:2.5 v/v/v) for 3 h followed by precipitation with cold Et₂O/pentane 3:1 v/v. Further workup (i.e. lyophilization and purification by cation chromatography) was performed according to the general procedure.

### Results

### Ub(1-76):

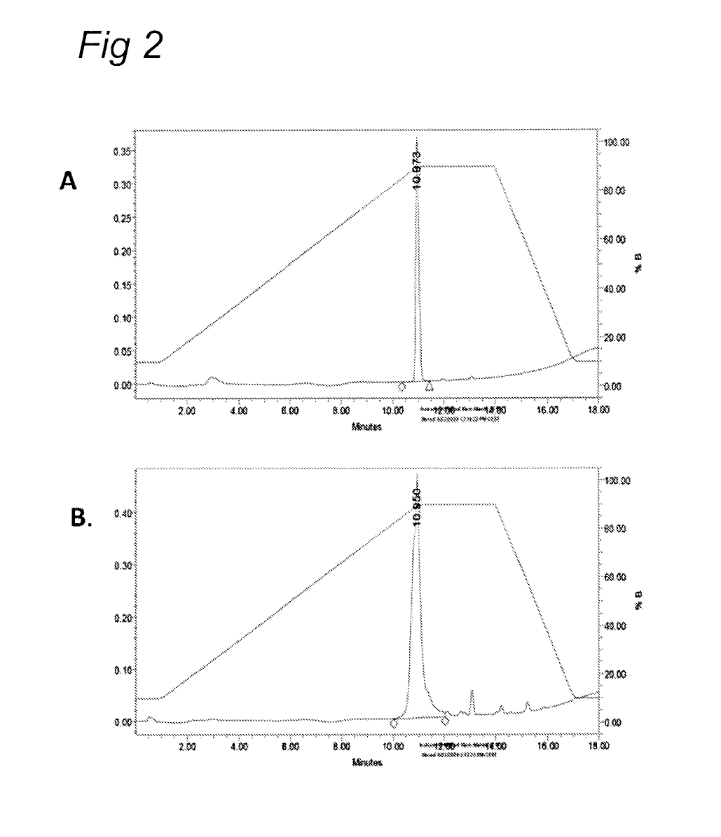

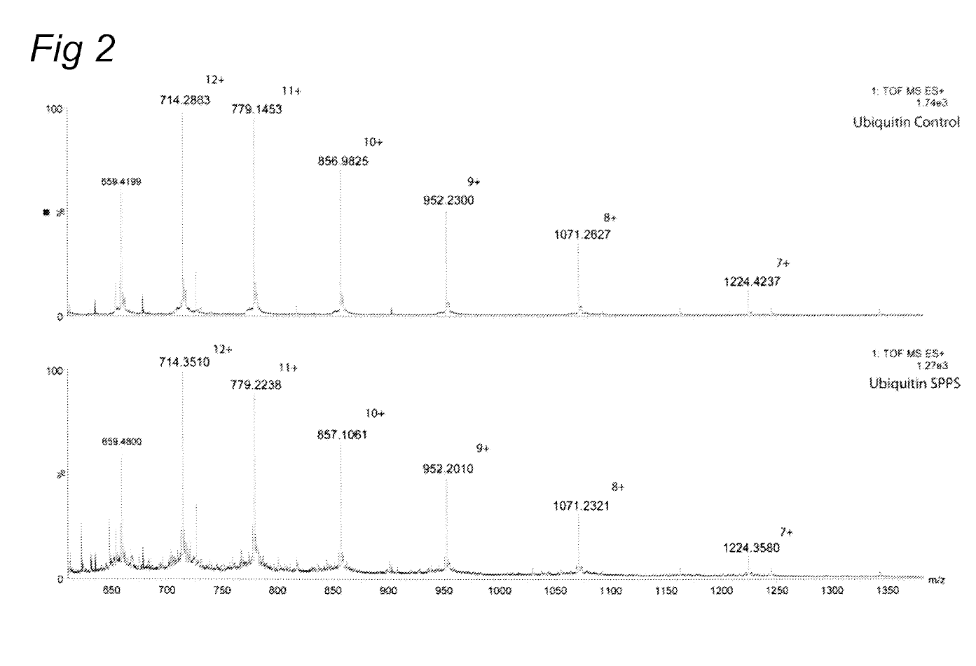

Crude yield of synthetic Ub(1-76) was 54%, yield of the purified product was 14%. LC-MS results, as shown in FIG. 2, confirm the identity of the synthetic product as Ub(1-76).

### His₆-Ub:

White powder (12.9 mg, 6%); LC-MS (program 3): Rₜ 2.18 min; MS ES+ (amu) calculated: 9388.19 [M+H]⁺. found 9388 [M+H]⁺.

### HA-Ub:

White powder (9.4 mg, 4%); LC-MS (program 3): Rₜ 2.38 min; MS ES+ (amu) calculated: 9649.5 [M+H]⁺. found 9649 [M+H]⁺.

UbK6δ-thiolysineG76V:

White powder (15.4 mg, 7.1%).

UhK11δ-thiolysineG76V:

White powder (16.2 mg, 7.5%).

UbK27δ-thiolysineG76V:

White powder (14.5 mg, 6.7%).

UbK29δ-thiolysineG76V:

White powder (19.1 mg, 8.8%).

UbK33δ-thiolysineG76V:

White powder (16.8 mg, 7.7%).

UhK48δ-thiolysineG76V:

White powder (18.6 mg, 8.6%).

UbK63δ-thiolysineG76

White powder (9.6 mg, 6.4%).

### CF-Ub:

The modification was carried out following the general procedure using resin-bound Ub(1-76) (12.5 μmol), 5(6)-carboxyfluorescein (47.0 mg, 125 μmol), DIC (19.4 μL, 125 μmol) and HOBt (16.9 mg, 125 μmol), in DMF (800 μL). The product (10.1 mg, 9%) was obtained as a bright yellow solid. LC-MS (program 3): Rₜ 2.40 min; MS ES+ (amu) calculated: 8923.7 [M+H]⁺. found 8924 [M+H]⁺.

### TAMRA-Ub:

The modification was carried out following the general procedure using resin-bound Ub(1-76) (12.5 μmol), TAMRA (53.8 mg, 125 μmol), DIC (19.4 μL, 125 μmol) and HOBt (16.9 mg, 125 μmol), in DMF (800 μL). The product (15.2 mg, 14%) was obtained as a deep purple solid. LC-MS (program 3): Rₜ 2.35 mm; MS ES+ (amu) calculated: 8977.8 [M+H]⁺. found 8978 [M+H]⁺.

### DOTA-Ub:

The modification was carried out following the general procedure using resin-bound Ub(1-76) (12.5 μmol), DOTA-tris-tert-butyl ester (71.6 mg, 125 mop, DIC (19.4 μL, 125 μmol) and HOBt (16.9 mg, 125 μmol), in DMF (800 μL). The product (10.5 mg, 9%) was obtained as a white solid. LC-MS (program 3): Rₜ 2.28 min; MS ES+ (amu) calculated: 8951.8 [M+H]⁺. found 8951 [M+H]⁺.

### Ub-VME:

The modification was carried out following the general procedure using resin-bound Ub(1-75) (25 μmol), (E)-methyl 4-aminobut-2-enoate 4-methylbenzenesulfonate (36 mg, 125 μmol), PyBOP (65 mg, 125 μmol) and TEA (52 μL, 375 μmol), in DCM (5 mL). The product (17.44 mg, 8%) was obtained as a white solid. LC-MS (program 3): Rₜ 2.27 min; MS ES+ (amu) calculated: 8605.5 [M+H]⁺. found 8605 [M+H]⁺.

### Ub-AMC:

The modification was carried out following the general procedure using resin-bound Ub(1-75) (25 μmol), 2-amino-N-(4-methyl-2-oxo-2H-chromen-7-yl)acetamide (58 mg, 250 μmol), PyBOP (65 mg, 125 μmol) and TEA (70 μL, 500 μmol), in DCM (5 mL). The product (13.8 mg, 6%) was obtained as a white solid. LC-MS (program 3): Rₜ 2.28 min; MS ES+ (amu) calculated: 8722.6 [M+H]⁺. found 8722 [M+H]⁺.

### Ub-Rh110-Gly:

The modification was carried out following the general procedure using resin-bound Ub(1-75) (25 μmol), glycine-rhodamine 110-glycine (112 mg, 250 μmol), PyBOP (65 mg, 125 μmol) and TEA (70 μL, 500 μmol), in DCM (5 mL). The product (10.47 mg, 5%) was obtained as a white solid. LC-MS (program 3): Rₜ 2.27 min; MS ES+ (amu) calculated: 8934.8 [M+H]⁺. found 8935 [M+H]⁺.

### Ub-SEt:

The modification was carried out following the general procedure using resin-bound Ub(1-75) (25 μmol), ethylmercaptane (92 μL, 1250 μmol), PyBOP (65 mg, 125 μmol) and TEA (209 μL, 1500 μmol), in DCM (5 mL). The product (15.3 mg, 7%) was obtained as a white solid. LC-MS (program 3): Rₜ 2.28 min; MS ES+ (amu) calculated: 8552.5 [M+H]⁺. found 8552 [M+H]⁺.

### EXAMPLE 2

### Structural Integrity and Folding of Synthetic Ub(1-76)

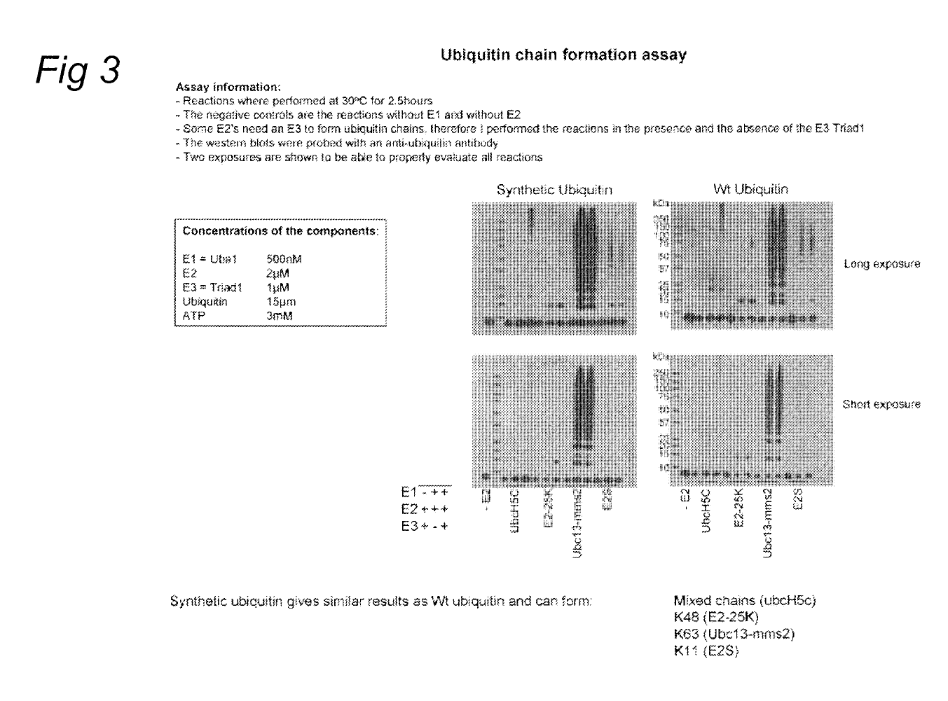

The structural integrity of the synthetic Ub(1-76) was tested by a polyubiquitination assay using UbE1, E2s (UbCH5c for mixed chains, E2-25K for K48 linked chains, Ubc13-mms2 for K63 linked chains, E2S for K11 linked chains) and the E3 Triad1 (performed by Judith Smith, B8, NKI-AVL). As can been seen in FIG. 3, the synthetic Ub is processed as native expressed Ub, confirming the structural integrity of the synthetic Ub polypeptide.

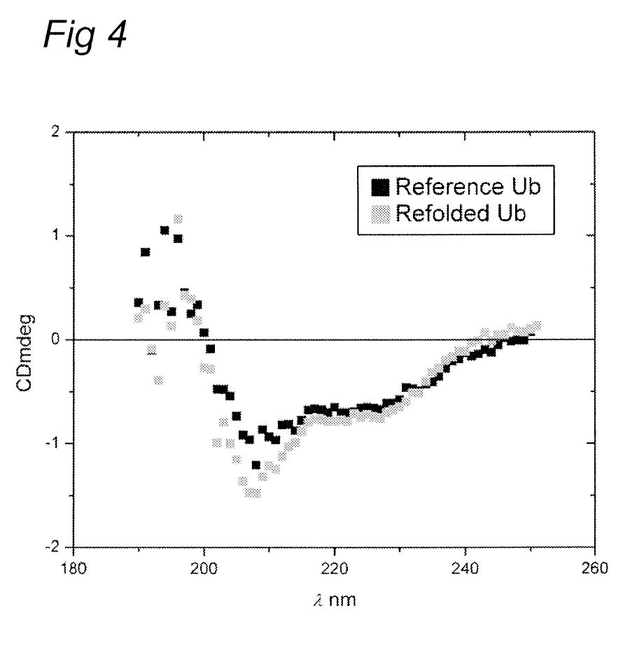

CD spectra were measured in 5 mM NH4OAc (pH 6.5) at a concentration of 0.5 mg/ml. Spectra were measured using a custom build machine with 0.5 mm optical path length. Data was obtained by averaging 20 scans. Step size was 1 nm with 2 sec. acquisition time. As can be seen in FIG. 4, correct folding of the synthetic Ubiquitin is confirmed with Circular dichroism measurement.

To further verify the correct folding of the synthetic Ub(1-76) a ligase assay was performed. E1, E2s and Triad1 E3 ligase were produced as described. Ubiquitin chain formation was assayed using 15 μM ubiquitin, 0.5 μM human Uba1 as E1, 2 μM E2 as mentioned, 3 mM ATP, in the presence and absence of 1 μM Triad1 as E3-ligase. Reactions were performed in 20 mM Hepes pH 7.5, 150 mM NaCl, 2 μM ZnCl₂, 10 mM MgCl₂, 2 mM DTT, for 2.5 hours at 30° C. and loaded onto 4-12% NuPage gel in MES-buffer.

### EXAMPLE 3

### Structural Integrity of Synthetic UbAMC and UbRh110Gly

Fully protected synthetic Ub(1-75), synthesized on hyper acid-labile trityl resin, was used in the syntheses of UbAMC and UbRh110Gly through condensation with GlyAMC and GlyRh110Gly respectively. Both Ub derivatives are routinely used to measure activity of deubiquitinating enzymes (DUBs). Upon coupling to GlyVME, fully protected synthetic Ub(1-75) was also converted into the active site directed probe Ub vinylmethyl ester (UbVME) which covalently modifies DUBs and as such can be used for DUB activity profiling. The structural integrity of synthetic UbAMC and UBRh110Gly, both prepared in accordance with the method of the present invention, was tested using the following assays.

Assay 1: Commercial and synthetic Ub-AMC, prepared according to the present method are treated side by side with HAUSP/USP7 and compared.

Assay 2. Synthetic Ub-AMC was treated with UCH-L3. It was found that the synthetic Ub-AMC was hydrolyzed and thus recognized as a substrate by the DUB. In this case we did not include AMC to determine the maximum emission, therefore, Vₘₐₓ was not calculated.

Assay 3: Ub-Rh110-Gly was treated with HAUSP/USP7 and UCH-L3. The synthetic Ub-Rh110-Gly was hydrolyzed and thus recognized as substrate by both DUBs. In this case we did not include Rh110-Gly to determine the maximum emission, therefore, Vₘₐₓ was not calculated.

### Determination of Concentrations of Ubiquitins for Biochemical Assays

Ubiquitins were dissolved in buffer and concentrations were determined by a Pierce 660 nM assay and mapped to a Ubiquitin standard curve.

| Ub mutant | Measured concentration |
|---|---|
| Fluorescein-Ub | 0.325 mg/ml |
| Ub-AMC | 0.563 mg/ml |
| Ub-TAMRA | 0.975 mg/ml |
| TAMRA-Ub | 0.438 mg/ml |

### Deubiquitinating Enzymes

UCH-L3 in pRSET vector was obtained from Dr. Keith Wilkinson. UCH-L3 was expressed in Escherichia coli and purified as described in: C. N. Larsen, J. S. Price, K. D. Wilkinson, K. D. Biochemistry 1996, 35, 6735-6744.

Usp7(206-1102) was expressed in E. coli from a synthetic construct and purified as described: (a) Shanmugham et al. J. Am. Chem. Soc. 2010, 132, 8834-8835; (b) Fernandez-Montalvan et al. Febs J. 2007, 274, 4256-1270.

### Ub-AMC/Ub-Rh110-Gly DUB Assay

assay buffer: 50 mM HEPES pH 7.5
100 mM NaCl
1 mM EDTA
0.05% Tween20
10 mM DTT
Reaction mixtures were incubated for 30 min at 25° C., measured every 5 minutes
30 μL reactions
Proteins used: Ub-AMC (Commercial)
   Ub-AMC (Synthetic)
   Ub-Rh110-Gly (Synthetic)
   USP7 full length
   UCH-L3
      All assays contained 1 nM DUB and the following concentrations of Ub:

| | 1 | 2 | 3 | 4 | 5 | 6 | 7 | 8 | 9 |
|---|---|---|---|---|---|---|---|---|---|
| Ub | 15 μM | 7.5 μM | 3.75 μM | 1.88 μM | 0.9 μM | 0.45 μM | 0.23 μM | 0.12 μM | 0.06 μM |

### Ubiquitin-AMC Assays

DUB activity on commercial (Sigma) and synthetic ubiquitin with a C-terminal fluorescent group, 7-amino-4-methylcoumarin (Ub-AMC) were performed at 25° C. in buffer containing 50 mM Hepes pH 7.5, 100 mM NaCl, 1 mM EDTA, 0.05% (w/v) Tween20, 10 mM DTT. The concentrations Ubiquitin-AMC and DUB are specified in the figures. Assays were performed in “Non binding surface flat bottom low flange” black 384-well plates (Corning) in 30 μl reactions. Kinetic data was collected in intervals of 5 min using a Fluostar Optima fluorescence plate reader (BMG Labtechnologies) at excitation and emission wavelengths of 355 nm and 460 nm, respectively for Ub-AMC. Experimental data was processed using Prism 4.03 (GraphPad Software, Inc.).

Binding of HAUSP/USP7 to ubiquitin-VME
Assay buffer: 20 mM HEPES pH7.5
150 mM NaCl
2 μM ZnCl₂
10 mM MgCl₂
2 mM DTT

Reaction mixtures were incubated for 60 min at 30° C.

10 μL reactions

Load 10 μL on gel (complete sample)

| | 1 | 2 | 3 |
|---|---|---|---|
| Ub-VME | 2 μM | 0 | 2 μM |
| USP7 | 0 | 1 μM | 1 μM |

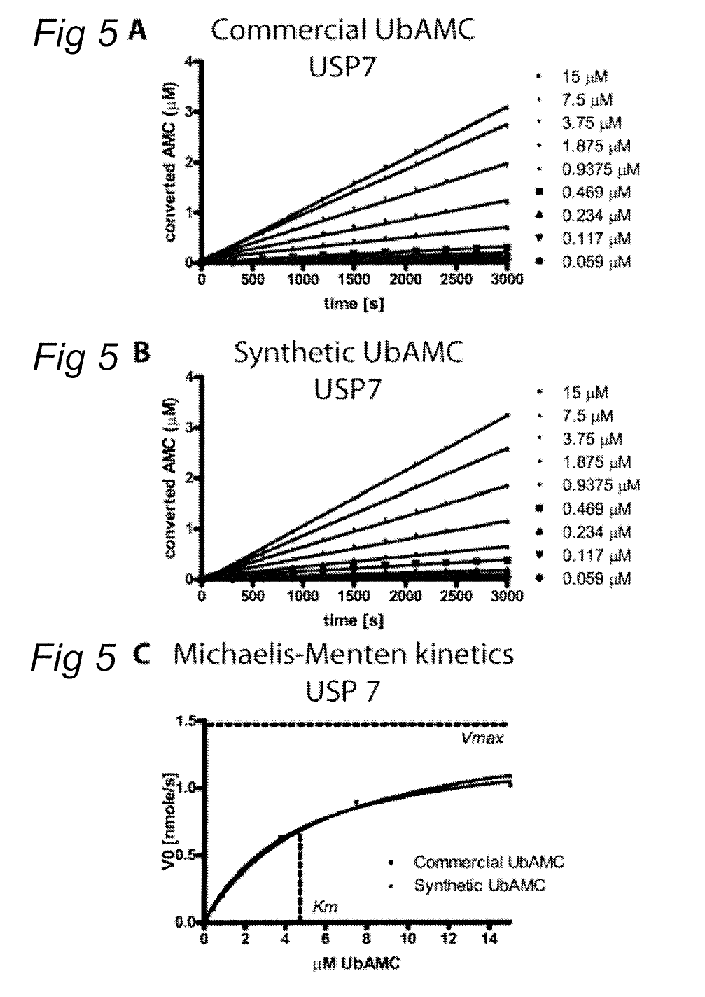

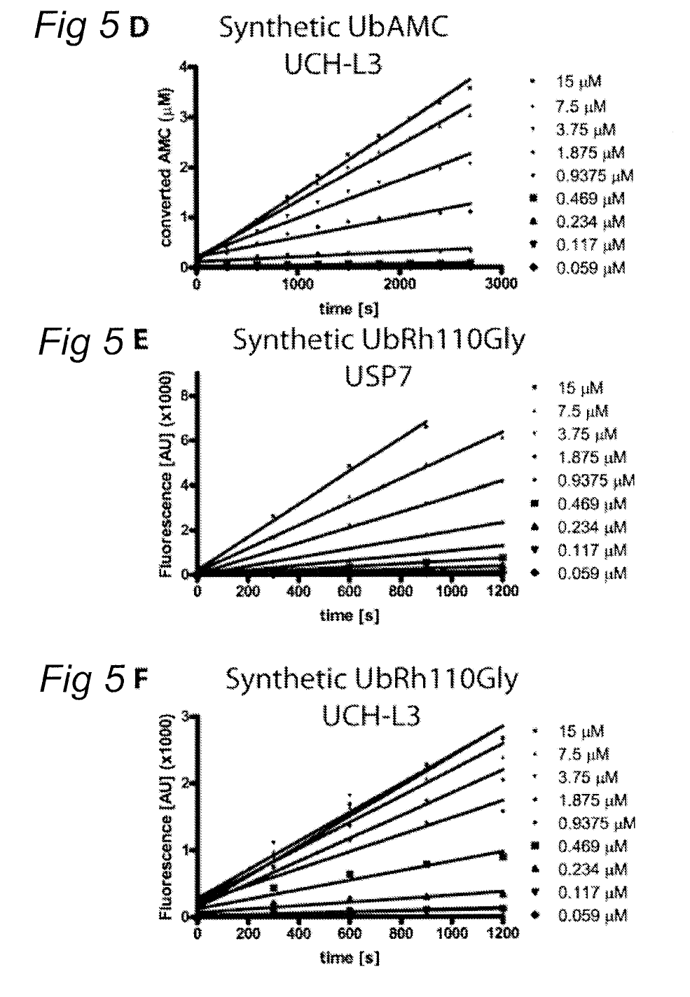

As can be seen in FIG. 5, structural integrity of synthetic UbAMC and UbRh110Gly was confirmed by their efficient turnover by the DUBs HAUSP/USP7 and UCH-L3. In the case of HAUSP/USP7, synthetic and commercial UbAMC showed comparable Kₘ values, indicating the same affinity for substrate and otherwise identical behaviour. The synthetic active-site targeted probe UbVME reacted swiftly as expected.

In conclusion, it has been shown that the present Fmoc-SPPS of Ub affords the desired product in high purity and yield. Various N-terminal fusions and various labels were incorporated successfully and mutant Ub's including lysine to d-thiolysine mutants were generated.

TABLE 1
Ubiquitin mutants of the invention

| | | |
|---|---|---|
| | | SEQ ID |
| Ub mutant | sequence | NO. |

Ub mutant that can not form polyUb chains

| | | |
|---|---|---|
| UbG76V | MQIFVKTLTGKTITLEVEPSDTIENVKAKIQDKEGIPPDQQRLIFAGKQLEDGRTLSDYNI | 2 |

Ub-chains by Native Chemical Ligation

| | | |
|---|---|---|
| UbG76C | MQIFVKTLTGKTITLEVEPSDTIENVKAKIQDKEGIPPDQQRLIFAGKQLEDGRTLSDYNI | 3 |
| UbM1C | CQIFVKTLTGKTITLEVEPSDTIENVKAKIQDKEGIPPDQQRLIFAGKQLEDGRTLSDYNI | 4 |

Ub with HA affinity tag; anti- HA staining

| | | |
|---|---|---|
| HA-Ub | YPYDVPDYAMQIFVKTLTGKTITLEVEPSDTIENVKAKIQDKEGIPPDQQRLIFAGKQLE | 5 |

Ub with His6 affinity tag; anti- His6 staining

| | | |
|---|---|---|
| His6-Ub | HHHHHHMQIFVKTLTGKTITLEVEPSDTIENVKAKIQDKEGIPPDQQRLIFAGKQLEDG | 6 |

Ub mutant with tag that enhances cell permeability

| | | |
|---|---|---|
| (D-Arg)8-Ub | rrrrrrrrMQIFVKTLTGKTITLEVEPSDTIENVKAKIQDKEGIPPDQQRLIFAGKQLEDGRT | 7 |
| Ub-(D-Arg)8 | MQIFVKTLTGKTITLEVEPSDTIENVKAKIQDKEGIPPDQQRLIFAGKQLEDGRTLSDYNI | 8 |
| Ub-penetratin | MQIFVKTLTGKTITLEVEPSDTIENVKAKIQDKEGIPPDQQRLIFAGKQLEDGRTLSDYNI | 9 |
| Penetratin-Ub | RQIKWFQNRRMQIFVKTLTGKTITLEVEPSDTIENVKAKIQDKEGIPPDQQRLIFAGKQ | 10 |
| Ub-Tat | MQIFVKTLTGKTITLEVEPSDTIENVKAKIQDKEGIPPDQQRLIFAGKQLEDGRTLSDYNI | 11 |
| Tat-Ub | YGRKKRRQRRRMQIFVKTLTGKTITLEVEPSDTIENVKAKIQDKEGIPPDQQRLIFAGK | 12 |

L4 handle for proteasome targeting

| | | |
|---|---|---|
| Ub-L4 | MQIFVKTLTGKTITLEVEPSDTIENVKAKIQDKEGIPPDQQRLIFAGKQLEDGRTLSDYNI | 13 |

Mutants for click chemistry Ligation via Huisgen [2 + 3} cycloaddition

| | | |
|---|---|---|
| UbM1(OrnN2) | (OrnN2)QIFVKTLTGKTITLEVEPSDTIENVKAKIQDKEGIPPDQQRLIFAGKQLEDGRTL | 14 |
| UbK6(OrnN2) | MQIFV(OrnN2)TLTGKTITLEVEPSDTIENVKAKIQDKEGIPPDQQRLIFAGKQLEDGRTL | 15 |
| UbK11(OrnN2) | MQIFVKTLTG(OrnN2)TITLEVEPSDTIENVKAKIQDKEGIPPDQQRLIFAGKQLEDGRTL | 16 |
| UbK27(OrnN2) | MQIFVKTLTGKTITLEVEPSDTIENV(OrnN2)AKIQDKEGIPPDQQRLIFAGKQLEDGRTL | 17 |
| UbK29(OrnN2) | MQIFVKTLTGKTITLEVEPSDTIENVKA(OrnN2)IQDKEGIPPDQQRLIFAGKQLEDGRTL | 18 |
| UbK33(OrnN2) | MQIFVKTLTGKTITLEVEPSDTIENVKAKIQD(OrnN2)EGIPPDQQRLIFAGKQLEDGRTL | 19 |
| UbK48(OrnN2) | MQIFVKTLTGKTITLEVEPSDTIENVKAKIQDKEGIPPDQQRLIFAG(OrnN2)QLEDGRTL | 20 |
| UbK63(OrnN2) | MQIFVKTLTGKTITLEVEPSDTIENVKAKIQDKEGIPPDQQRLIFAGKQLEDGRTLSDYNI | 21 |

Mutants for native chemical ligation Ub-chains by Native Chemical Ligation

| | | |
|---|---|---|
| UbK6(δ- | MQIFV(δ- | 22 |
| UbK11(δ- | MQIFVKTLTG(δ- | 23 |
| UbK27(δ- | MQIFVKILIGKTITLEVEPSDTIENV(δ- | 24 |
| UbK29(δ- | MQIFVKTLTGKTITLEVEPSDTIENVKA(δ- | 25 |
| thioK)G76V | thioK)IQDKEGIPPDQQRLIFAGKQLEDGRTLSDYNIQKESTLHLVLRLRGV | |
| UbK33(δ- | MQIFVKTLTGKTITLEVEPSDTFIENVKAKIQD(δ- | 26 |
| UbK48(δ- | MQIFVKTLTGKTITLEVEPSDTIENVKAKIQDKEGIPPDQQRLIFAG(δ- | 27 |
| UbK63(δ- | MQIFVKTLTGKTITLEVEPSDTIENVKAKIQDKEGIPPDQQRLIFAGKQLEDGRTLSDYNI | 28 |

Mutants for native chemical ligation

| | | |
|---|---|---|
| UbK6(δ-thioK) | MQIFVKTLTG(δ- | 29 |
| UbK11(δ-thioK) | MQIFVKILTG(δ- | 30 |
| UbK27(δ-thioK) | MQIFVKTLTGKTITLEVEPSDTIENV(δ- | 31 |
| UbK29(δ-thioK) | MQIFVKTLTGKTITLEVEPSDTIENVKA(δ- | 32 |
| UbK33(δ-thioK) | MQIFVKTLTGKTITLEVEPSDTIENVKAKIQD(δ- | 33 |
| UbK48(δ-thioK) | MQIFVKTLTGKTITLEVEPSDTIENVKAKIQDKEGIPPDQQRLIFAG(δ- | 34 |
| UbK63(δ-thioK) | MQIFVKTLTGKTITLEVEPSDTIENVKAKIQDKEGIPPDQQRLIFAGKQLEDGRTLSDYN | 35 |
| UbK48(γ-thioK) | MQIFVKTLIGKTITLEVEPSDTIENVKAKIQDKEGIPPDQQRLIFAG(γ- | 36 |

Mutants for crosslinking studies

| | | |
|---|---|---|
| UbL43photoLeu | MQIFVKTLTGKTITLEVEPSDTIENVKAKIQDKEGIPPDQQR(photoLeu)IFAGKQLEDG | 37 |
| UbL7lphotoLeu | MQIINKTLTGKTITLEVEPSDTIENVKAKIQDKEGIPPDQQRLIFAGKQLEDGRTLSDYN | 38 |
| UbL73photoLeu | MQIFVKTUFGKTITLEVEPSDTIENVKAKIQDKEGIPPDQQRLIFAGKQLEDGRILSDYN | 39 |

TABLE 2
Ubiquitin derivatives of the invention

| Ub | Sequence | Function |
|---|---|---|
| CF-Ub | CF- | fluorogenic Ub (absorption @ |
| TAMRA- | TAMRA- | fluorogenic Ub (absorption @ |
| DOTA-Ub | DOTA- | Ub with chelate, complex |
| Ub-PA | MQIFVKILTGKTITLEVEPSDTIENVKAKIQDKEGIPPDQQRLIFAGKQLEDG | Ligation via Huisgen [2 + 3} |
| Ub-VME | MQIFVKTLTGKTITLEVEPSDTIENVKAKIQDKEGIPPDQQRLIFAGKQLEDG | |
| Ub-AMC | MQIFVKTLTGKTITLEVEPSDTIENVKAKIQDKEGIPPDQQRLIFAGKQLEDG | Fluorogenic Ub, essay reagent |
| Ub-SEt | MQIFVKILTGKTITLEVEPSDTIENVKAKIQDKEGIPPDQQRLIFAGKQLEDG | Ub thioester for Native Chemical |
| Ub- | MQIFVKILTGKTITLEVEPSDTIENVKAKIQDKEGIPPDQQRLIFAGKQLEDG | Fluorogenic Ub, essay reagent |

### Synthesis of δ-thiolysine

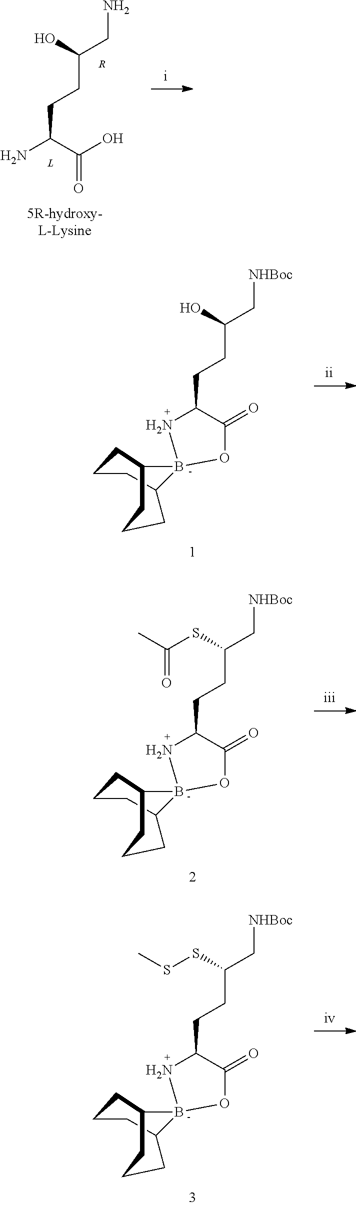

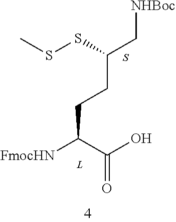

A 28% ammonia solution (aq. 10 mL) was added to (5R)-5-hydroxy-L-lysine dihydrochloride monohydrate (1.04 g, 4.11 mmol) at 0° C. After stirring for 30 min the solution was concentrated and the crystalline solid was dried in high vacuum before further use. The solid was added in one portion to a stirred solution of 9-BBN (1.2 g, 4.7 mmol) in hot methanol (20 mL). The reaction mixture was refluxed (ca. 3 h) under N₂ until a clear solution was obtained. After evaporation of the solvent, the residue was dissolved in 1,4-dioxane/water (⅔ v/v, 30 mL), cooled in an ice-bath and treated with NaHCO₃ (0.5 g) and Boc₂O (1.1 g). After stirring overnight, the reaction mixture was concentrated, diluted with brine and extracted with EtOAc. After drying (MgSO₄) and concentration, the crude product was purified over silica gel (n-hexane/EtOAc; 1/0→0/1). Compound 1 (R_{f}=0.4, EtOAc) was obtained as a white foam. Yield: 1.39 g, 3.63 mmol, 89% over 2 steps. On a 45.7 mmol scale, the product was obtained (silica gel chromatography DCM→>10% MeOH/DCM) in an overall yield of 82%.

To a solution of 1 (1.35 g, 2.48 mmol) and Et₃N (730 μL, 5.24 mmol) at 0° C. in dichloromethane (15 mL) was added MSCl (326 μL, 4.19 mmol). The reaction mixture was stirred for 1 hour when TLC analysis showed completion. The crude product was purified over silica gel (n-hexane/EtOAc 1:1→1:3) affording the mesylate (R_{f}=0.8, EtOAc) as a foam. Yield: >99%. ¹H-NMR (400 MHz, MeOD-d₄) δ 6.40 (m, 1H), 5.83 (m, 1H), 4.98 (m, 1H), 4.69 (m, 1H), 3.70 (m, 2H, H-α and H-δ), 3.25-3.12 (m, partially obscured by MeOD-d₄ peak), 2.69 (s, 3H), 2.11 (m, 1H, H-β), 2.12-1.30 (m, CH-boron, H-β and H-γ), 1.20 (s, 9H, tBu Boc), 0.57 (broad s, 2H, CH₂ boron). LC-MS (program 1): Rₜ=7.3 min, MS ES+(amu): 461.19 [M+H]⁺, 920.77 [M-M+H]⁺. Potassium acetate (1.75 eq, 10.9 mmol, 1.25 g) was added to a solution of the mesylate (1.0 eq, 2.87 g, 6.23 mmol) in dry DMF (58 mL). The reaction was stirred at 65° C. for 3 hrs when TLC and LC-MS analysis showed completion. The DMF was evaporated and the concentrate was dissolved in EtOAc, washed with water and brine, dried, and concentrated. Yield after silica gel chromatography: 2.21 g, 5.05 mmol, 81%.

Thioacetate 2 (1.13 g, 2.5 mmol) was dissolved in methanol (15 mL) and treated with 1N NaOH solution (3 mL) for 15 min at 0° C. The reaction mixture was carefully neutralized with equimolar amounts of HOAc and concentrated. The concentrate was dissolved in ethyl acetate and washed with water and brine, dried (MgSO₄), and concentrated affording the crude thiol as an oil. ¹H-NMR (400 MHz, MeOD-d₄) 83.64 (app t, 1H, H-α J=7.5 and 5.4 Hz), 3.28 (dd, 1H, H-ε, J=7.6 and 14.1 Hz), 3.13 (dd, 1H, H-ε′, J=6.8 and 13.9 Hz), 2.87 (broad s, 1H, H-δ), 2.10-1.30 (m, CH-boron, H-β and H-γ), 1.43 (s, 9H, tBu Boc), 0.57 (broad d, 2H, CH₂ boron, J=13.9 Hz). ¹³C-NMR (100 MHz, MeOD-d₄) δ 177.3 (C═O), ˜159 (C═O, Boc, low intensity peak), 80.5 (C_{q} tBu), 56.2 (CH), 49.1 (CH₂, partially obscured by MeOD-d₄). 41.5 (CH), 33.1, 32.8, 32.7, 32.5, 32.4, 29.7 (5×CH₂), 29.0 (CH), 28.9 (tBu, Boc), 25.8, 25.4 (2×CH₂). Next, a degassed solution of the thiol in DCM (7 mL) was added dropwise to a degassed solution of S-Methyl methanethiosulfonate (3 equiv, 6.9 mmol, 0.66 mL) and Et₃N (9 equiv, 2.76 mL, 20.4 mmol) in DCM (7 mL). The reaction mixture was stirred for 1 h when TLC analysis (n-hexane/EtOAc 1:3 v/v) showed completion. After evaporation of the DCM, the crude product was purified over silica gel (n-hexane/EtOAc 2:3 v/v) affording 3 (R_{f}=0.8, EtOAc) as an oil. Yield: 1.1 g, 2.5 mmol, >99% over 2 steps.

Compound 3 (2.24 g, 5.0 mmol) was dissolved in THF (40 mL) and ethylene diamine (1.4 mL) was added. When the solution was heated (oil-bath ˜70° C. or heatgun) a white solid precipitated (9-BBN.ethylene diamine complex). The reaction mixture was cooled and the precipitate filtered over Hyflo®). The filtrate was concentrated and in case of more precipitate being formed, filtered again. Flash column chromatography (DCM→40% MeOH in DCM, R_{f}=0.4) gave IV-tert-butoxycarbonyl-5S-(methyldisulfanyl)-L-lysine as a gummy solid (1.27 g, 3.91 mmol, 78%). LC-MS (program 1): R₁=5.7 min, ES+ (amu): 325.40 [M+H]⁺. 649.39 [M-M+H]. Next, a solution of Fmoc-OSu (1.25 eq, 1.65 g, 4.881 mmol) in acetone (25 mL) was added to a cooled solution of N^{ε}-tert-Butoxycarbonyl-5S-(methyldisulfanyl)-L-lysine (1.27 g, 3.91 mmol) and NaHCO₃ (360 mg, 4.30 mmol, 1.1 eq) in acetone/H₂O (225 mL/50 mL). The reaction mixture was stirred overnight, analysed by TLC/LCMS, concentrated, acidified with 1N aq. KHSO₄ and extracted with EtOAc. The organic layer was dried (MgSO₄) and concentrated. Silica gel chromatography (0→10% MeOH in DCM) gave 4 as an oil which formed a foam under high vacuum. Yield: 2.1 g, 3.9 mmol, 99%.

### Synthesis of Fmoc-photo-Leu-OH

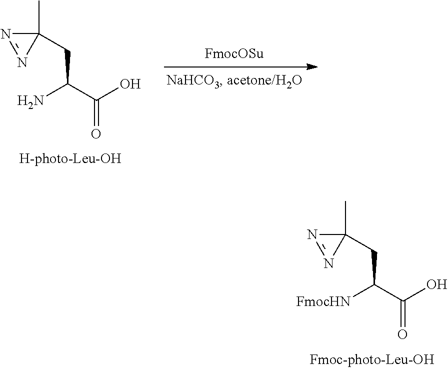

A solution of Fmoc-OSu (1.2 eq, 2.5 mmol, 850 mg) in acetone (25 mL) was added to a solution of H-photoLeu-OH (300 mg, 2.1 μmol, ThermoScientific) in 10% aq. NaHCO₃ (25 mL). The reaction mixture was stirred overnight at rt and analysed by LC-MS. The reaction mixture is concentrated for 50% and washed with Et₂O. The basic aq. layer is acidified to pH ≈1 with 1N aq. Hcl. The product can then be extracted with EtOAc. Upon acidification, the product precipitates after which it can be isolated by filtration. Yield: >99%. LC-MS Rₜ 9.9 min; MS ES (amu) calculated: 387.36 [M+Na]⁺. found 387.98 [M+Na]⁺. Waters Atlantis T3™ C18 (2.1×100 mm, 3 μM); flow rate=0.4 mL/min, runtime=20 min, column T=40° C. Gradient: 5% [symbol] 95% B over 16 min.

Synthesis γ-thiolysine

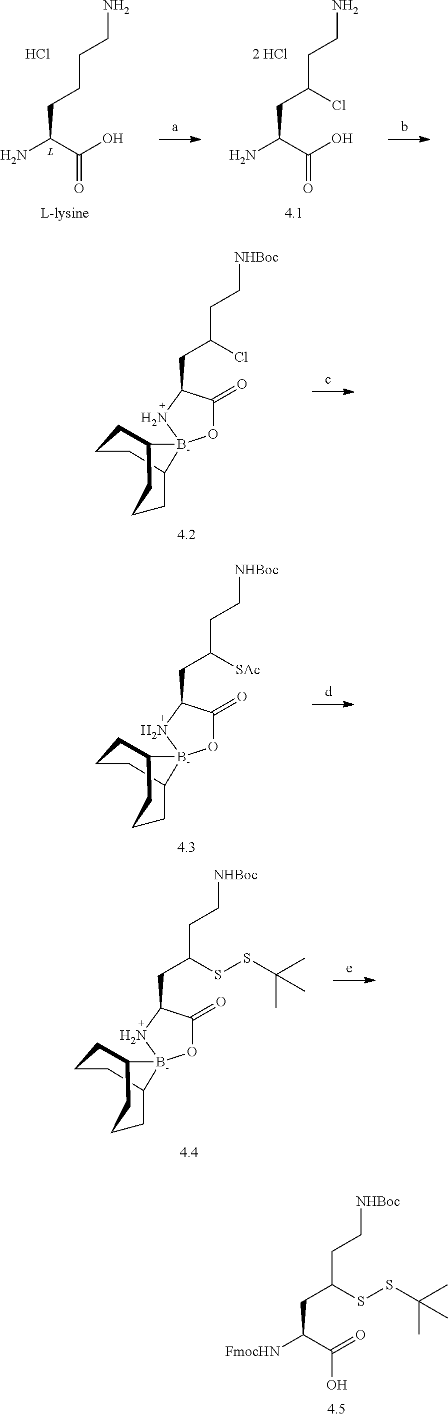

The synthesis of 4.1 was performed following the literature procedure as was described by Kollonitsch et al (J. Am. Chem. Soc. 1964, 86, 1857). Chlorine was bubbled through a solution of L(+)-lysine monohydrochloride (150 g, 821 mmol) in HCl (36%) at 70° C. The mixture was irradiated with a medium pressure mercury lamp while stirring. After 2 h the reaction mixture was cooled to 7° C. and a seed crystal was added to induce crystallization. After one hour the resulting crystals were filtered off and the crude product was triturated twice with MeOH. The crystals were collected to afford 4.1 as a white solid.

DiPEA (1.75 mL, 10.0 mmol) was added to a stirred solution of 4.1 (1.27 g, 5.0 mmol) in dry MeOH (25 mL). The reaction mixture turned turbid and after 5 minutes 9-BBN (1.40 g, 5.75 mmol) was added. The suspension was heated at 70° C. under nitrogen until a clear solution was obtained (approx. 2 h). LC-MS analysis confirmed complete conversion to the boronated product: R₁=6.73 min (LC-MS program 1), MS ES+ (amu): 300.98 [M+H]⁺). The solvent was removed in vacuo and the residue was coevapporated twice with DCM. The residue was dissolved in dry THF (25 mL) and DiPEA (1.75 mL, 10.0 mmol) and Boc₂O (1.091 g, 5.0 mmol) were added. The reaction mixture was stirred for 3 h before 1N KHSO₄ (25 mL) was added. The THF was removed in vacuo, and the remaining aqueous phase was extracted with EtOAc. Subsequently, the organic layer was washed with 1N KHSO₄ and brine, dried (Na₂SO₄) and concentrated. The product was isolated as a white foam by flash column chromatography (EtOAc/n-hexane 3/7→1/1 v/v).

KSAc (122 mg, 1.07 mmol) was added to a solution of 4.2 (244 mg, 0.61 mmol) in DMF (10 mL). The reaction mixture was stirred at 65° C. for 3 h before the solvent was removed in vacuo. The residue was redissolved in EtOAc, washed with brine, dried (Na₂SO₄) and concentrated. The product was isolated as a white foam by flash column chromatography (EtOAc/n-hexane 3/7→1/1 v/v).

Thioacetate 4.3 (597 mg, 1.36 mmol) was dissolved in methanol (14 mL) and treated with 1N NaOH (1.36 mL) for 30 min at 0° C. The reaction mixture was carefully neutralized by the addition of equimolar amounts of HOAc and concentrated. The concentrate was redissolved in ethyl acetate and washed with 1N KHSO₄ and brine, dried (Na₂SO₄), and concentrated affording the crude thiol as an oil. In a separate flask, a mixture of MsCl (0.53 mL, 6.80 mmol), 2-methyl-2-propanethiol (0.767 mL, 2.72 mmol) and Et₃N (1.90 mL, 13.6 mmol) in DCM (25 mL) was stirred for 30 min before a solution of the crude thiol and Et₃N (0.190 mL, 1.36 mmol) in DCM (25 mL) was added. The reaction mixture was stirred for an additional 2 h. Next, 1N KHSO₄ (50 mL) was added and the DCM was removed in vacuo. The aqueous residue was extracted with EtOAc and the organic layer was washed with 1N KHSO₄ and brine, dried (Na₂SO₄) and concentrated. The product was isolated as a white foam by flash column chromatography (DCM→EtOAc/DCM 1/1 v/v).

2N LiOH (7.5 mL) was added to a solution of 4.4 (243 mg, 0.5 mmol) in THF (7.5 mL) and was stirred vigorously for 2 h before the THF was removed in vacuo. The aqueous residue was acidified to pH=4 with 1N HCl, and washed with DCM. The water layer was concentrated to 25 mL and the pH was brought to 8.5 with Et₃N. A solution of Fmoc-OSu (252 mg, 0.75 mmol) in MeCN (25 mL) was added. The reaction mixture was stirred at room temperature while the pH was kept between 8 and 8.5. After 30 min the reaction mixture was acidified to pH=3 with 1N HCl and the MeCN was removed in vacuo. 1N KHSO₄ (25 mL) was added and the mixture was extracted with EtOAc. The organic layer was washed with 1N KHSO₄ and brine, dried (Na₂SO₄) and concentrated. The product was isolated as a white foam by flash column chromatography (5% MeOH in DCM→10% MeOH in DCM v/v %).

1.-17. (canceled)

18. A method of preparing a peptide selected from the group consisting of ubiquitin, a ubiquitin mutant and a derivative thereof, the method comprising:
(a) synthesizing the peptide on a solid phase by stepwise coupling of Fmoc-protected, optionally further suitably side-chain protected, amino acids, dipeptides and/or oligopeptides in a linear C-terminal to N-terminal fashion; and subsequently,
(b) cleaving the peptide from the solid phase and deprotecting the peptide;
wherein, in step (a), at least four amino acid pairs are added during synthesis in the form of a building block, wherein the amino acid pairs are separated from each other by at least two amino acids and are selected from the pairs at positions 6-7; 8-9; 11-12; 13-14; 21-22; 46-47;
and 52-53 of ubiquitin (SEQ ID no. 1) or from corresponding pairs of a ubiquitin mutant sequence.

19. The method according to claim 18, wherein each amino acid pair added as an amide protected building block is separated from any proline residue by at least 4 amino acids.

20. The method according to claim 18, wherein, in step (a), at least five amino acid pairs are added during synthesis in the form of a building block, wherein the amino acid pairs are separated from each other by at least two amino acids and are selected from the pairs at positions 6-7; 8-9; 11-12; 13-14; 21-22; 46-47; 52-53; 56-57; and 65-66 of ubiquitin sequence (SEQ ID no. 1) or from corresponding pairs of a ubiquitin mutant sequence.

21. The method according to claim 18, wherein, in step (a), at least six amino acid pairs are added during synthesis in the form of a building block, wherein the amino acid pairs are separated from each other by at least two amino acids and are selected from the pairs at positions 6-7; 8-9; 11-12; 13-14; 21-22; 46-47; 52-53; 56-57; and 65-66 of ubiquitin sequence (SEQ ID no. 1) or from corresponding pairs of a ubiquitin mutant sequence.

22. The method according to claim 18, wherein, in step (a), amino acid pairs at positions 8-9; 13-14; 46-47; 52-53; 56-57; and 65-66 of ubiquitin sequence (SEQ ID no. 1) or the corresponding pairs in a ubiquitin mutant sequence are added during synthesis in the form of a building block.

23. The method according to claim 18, wherein the building blocks are independently selected from the group consisting of pseudoproline (oxazolidine) dipeptides, dimethoxybenzyl dipeptides and isoacyl dipeptides.

24. The method according to claim 18, wherein the amide protected building blocks are independently selected from the group consisting of Fmoc-Leu-Thr(ψMe,Mepro)-OH; Fmoc-Ile-Thr(ψMe,Mepro)-OH; Fmoc-Ala-(Dmb)-Gly-OH; Fmoc-Lys(Boc)-Thr(ψMe,Mepro)-OH; Fmoc-Asp(OtBu)-Thr(ψMe,Mepro)-OH; Fmoc-Asp(OtBu)-(Dmb)-Gly-OH; Fmoc-Leu-Ser(ψMe,Mepro)-OH; and Fmoc-Glu(OtBu)-Ser(ψMe,Mepro)-OH; Fmoc-Ser(tBu)-Thr(ψMe,Mepro)-OH.

25. The method according to claim 18, wherein the synthesizing comprises addition of:
(i) Fmoc-Leu-Thr(ψMe,Mepro)-OH at amino acid positions 8-9;
(ii) Fmoc-Ile-Thr(ψMe,Mepro)-OH at amino acid positions 13-14;
(iii) Fmoc-Ala-(Dmb)-Gly-OH at amino acid positions 46-47;
(iv) Fmoc-Asp(OtBu)-(Dmb)-Gly-OH at amino acid positions 52-53;
(v) Fmoc-Leu-Ser(ψMe,Mepro)-OH at amino acid positions 56-57; and/or
(vi) Fmoc-Ser(tBu)-Thr(ψMe,Mepro)-OH at amino acid positions 64-65, of ubiquitin sequence (SEQ ID no. 1) or at the corresponding positions of a ubiquitin mutant sequence.

26. The method according to claim 18, comprising stepwise coupling of Fmoc-protected, optionally further suitably side-chain protected, amino acids and/or dipeptides.

27. The method according to claim 18, comprising: (c) purifying the peptide of (b) using cation chromatography.

28. The method according to claim 18, wherein the synthesizing comprises ligating of a ligand to a reactive amino acid side chain and/or the N-terminal amine moiety of the peptide.

29. The method according to claim 18, comprising ligating of a ligand to the C-terminal carboxyl group of the peptide following (b).

30. The method according to claim 18, comprising removing the protective groups from the peptide following step (a) or (b).

31. The method according to claim 28, wherein the ligand is selected from the group consisting of fluorophores, affinity labels, biophysical labels, chelating agents, complexing agents, and epitope tags,

32. The method according to claim 31, wherein the ligand is selected from the group consisting of fluorescin, TAMRA, DOTA, propargylamine, VME, AMC and SEt.

33. The method according to claim 28, wherein the ligand is another peptide.

34. The method according to claim 28, wherein the other peptide is another ubiquitin or ubiquitin mutant.

35. The method according to claim 18, comprising: (c) folding of the peptide and/or purification of the peptide.

36. A peptide selected from the group consisting of ubiquitin, ubiquitin mutants and derivatives thereof, wherein at least four amino acid pairs of the peptide are been replaced with a corresponding building block, wherein the amino acid pairs are separated from each other by at least two amino acids and are selected from the pairs at positions 6-7; 8-9; 11-12; 13-14; 21-22; 46-47; and 52-53 of the ubiquitin sequence (SEQ ID no. 1) or from corresponding pairs of a ubiquitin mutant sequence.
